SEQUENCE LISTING (23 sequences)

SEQ ID NO: 1 - protein, 5 aa, Artificial Sequence
  Synthetic Construct
GGGGG

SEQ ID NO: 2 - protein, 5 aa, Artificial Sequence
  Synthetic Construct
GGGGS

SEQ ID NO: 3 - protein, 10 aa, Artificial Sequence
  Synthetic Construct
GGGGSGGGGS

SEQ ID NO: 4 - protein, 15 aa, Artificial Sequence
  Synthetic Construct
GGGGSGGGGSGGGGS

SEQ ID NO: 5 - protein, 20 aa, Artificial Sequence
  Synthetic Construct
GGGGSGGGGSGGGGSGGGGS

SEQ ID NO: 6 - protein, 30 aa, Artificial Sequence
  Synthetic Construct
GGGGSGGGGSGGGGSGGGGSGGGGSGGGGS

SEQ ID NO: 7 - protein, 5 aa, Artificial Sequence
  Synthetic Construct
SSSSG

SEQ ID NO: 8 - protein, 10 aa, Artificial Sequence
  Synthetic Construct
SSSSGSSSSG

SEQ ID NO: 9 - protein, 15 aa, Artificial Sequence
  Synthetic Construct
SSSSGSSSSGSSSSG

SEQ ID NO: 10 - protein, 20 aa, Artificial Sequence
  Synthetic Construct
SSSSGSSSSGSSSSGSSSSG

SEQ ID NO: 11 - protein, 25 aa, Artificial Sequence
  Synthetic Construct
SSSSGSSSSGSSSSGSSSSGSSSSG

SEQ ID NO: 12 - protein, 30 aa, Artificial Sequence
  Synthetic Construct
SSSSGSSSSGSSSSGSSSSGSSSSGSSSSG

SEQ ID NO: 13 - protein, 12 aa, Artificial Sequence
  Synthetic Construct
GKSSGSGSESKS

SEQ ID NO: 14 - protein, 15 aa, Artificial Sequence
  Synthetic Construct
GGSTSGSGKSSEGKG

SEQ ID NO: 15 - protein, 19 aa, Artificial Sequence
  Synthetic Construct
GSTSGSGKSSSEGSGSTKG

SEQ ID NO: 16 - protein, 18 aa, Artificial Sequence
  Synthetic Construct
GSTSGSGKPGSGEGSTKG

SEQ ID NO: 17 - protein, 14 aa, Artificial Sequence
  Synthetic Construct
EGKSSGSGSESKEF

SEQ ID NO: 18 - protein, 4 aa, Artificial Sequence
  Synthetic Construct
AAPA

SEQ ID NO: 19 - protein, 8 aa, Artificial Sequence
  Synthetic Construct
AAPAAAPA

SEQ ID NO: 20 - protein, 12 aa, Artificial Sequence
  Synthetic Construct
AAPAAAPAAAPA

SEQ ID NO: 21 - protein, 16 aa, Artificial Sequence
  Synthetic Construct
AAPAAAPAAAPAAAPA

SEQ ID NO: 22 - protein, 20 aa, Artificial Sequence
  Synthetic Construct
AAPAAAPAAAPAAAPAAAPA

SEQ ID NO: 23 - protein, 24 aa, Artificial Sequence
  Synthetic Construct
AAPAAAPAAAPAAAPAAAPAAAPA

(19) United States
(12) Patent Application Publication
(10) Pub. No.: US 2010/0278801 A1
(43) Pub. Date: Nov. 4, 2010

(54) COMPOSITIONS COMPRISING OPTIMIZED HER1 AND HER3 MULTIMERS AND METHODS OF USE THEREOF

(76) Inventors: H. Michael Shepard, San Francisco, CA (US); Pei Jin, Palo Alto, CA (US)

Correspondence Address: MORRISON & FOERSTER LLP, 755 PAGE MILL RD, PALO ALTO, CA, 94304-1018, US

(21) Appl. No.: 12/682,584

(22) PCT Filed: Oct. 15, 2008
(86) PCT No.: PCT/US08/79998

§ 371 Date: Jun. 21, 2010

Related U.S. Application Data
(60) Provisional application No. 60/980,424, filed on Oct. 16, 2007.
(60) Provisional application No. 61/043,308, filed on Apr. 8, 2008.

(51) Int. Cl. A61K 38/45; C12N 9/12; C12N 9/96; A61P 35/00

(52) U.S. Cl. 424/94.5; 435/194; 435/188

(57) ABSTRACT

The invention provides for compositions comprising engineered Her3 multimers with improved binding affinity. Such multimers include, but are not limited to, Her1/Her 3 heterodimers in which the Her3 ligand binding domain has been optimized to increase binding to Her3. The composition also can include mixtures of Her 1 homodimers, Her 3 homodimers, and Her 1/Her 3 heterodimers.

### CROSS-REFERENCES TO RELATED APPLICATIONS

This application claims priority benefit to U.S. provisional applications 60/980,424, filed on Oct. 16, 2007, and 61/043,308, filed on Apr. 8, 2008, both of which are incorporated by reference herein in its entirety.

### FIELD OF THE INVENTION

The invention relates to compositions that comprise engineered Her 1 and Her3 ligand binding domains and to methods of making and using such compositions.

### BACKGROUND

Receptor tyrosine kinases (RTKs) are a family of cell signaling molecules that are among the polypeptides involved in many signal transduction pathways. RTKs play a role in a variety of cellular processes, including embryogenesis, cell division, proliferation, differentiation, migration and metabolism. RTKs can be activated by ligands. Such activation, in turn, usually results in receptor dimerization or oligomerization as a requirement for the subsequent activation of the signaling pathways. Activation of the signaling pathway, such as by triggering autocrine or paracrine cellular signaling pathways, for example, activation of second messengers, results in specific biological effects. Ligands for RTKs specifically bind to the cognate receptors. Disregulation of RTKs has been noted in several cancers. For example, breast cancer can be associated with amplified expression of p185-HER2. RTKs also are associated with regulating pathways involved in angiogenesis, including physiologic and tumor blood vessel formation. RTKs also are implicated in the regulation of cell proliferation, migration and survival.

Among the RTKs associated with disease is the HER (Human EGFR family, also referred to as the ErbB or EGFR) family of receptors (see, e.g., Hynes et al. (2005) Nature Reviews Cancer 5:341-354, for a discussion of their role cancer). These receptors, referred to as the Class I receptors, include HER1/EGFR, HER2, HER3 and HER4. These receptors have alternate names. HER1 is sometimes referred to as EGFR and ErbB1; HER2 is referred to sometimes as ErbB2 and NEU; HER3 is referred to sometimes as ErbB3; and HER4 is referred to sometimes as ErbB4. All members of this family have an extracellular ligand-binding region, a single membrane-spanning region and a cytoplasmic tyrosine-kinase-containing domain. Only HER1 and HER4 are fully functional in terms of ligand binding and kinase activity. HER3 has impaired kinase activity and relies on the kinase activity of its heterodimerization partners for activation.

One approach for targeting cancer involving p185 (Her2) has been to use peptides targeting the ErbB2 protein dimers (see, for example, Greene at al., U.S. Pat. No. 6,417,168). Various types of chimeric multimeric molecules that include the ligand binding domains for ErbB2, Erb3 and ErbB4 have been described. See, for example, U.S. Pat. No. 6,696,290 and WO 98/02540. However, these approaches are neither specific to cancers which have expression of Her 1 and/or Her3, nor can they reach a broader spectrum of diseases associated with dysregulation of Her1 and/or Her3 expression. As such, for the cancers which have dysregulation of Her1 or dysregulation or Her3 or a combination of the two, the existing technology is not sufficient to overcome the problem of not having enough specificity. In addition, the binding affinity for ligands for these Class I receptors varies depending on the receptor and its inherent biology and structure. Accordingly, a molecule which binds to ErbB2 will not necessarily bind to Her1 or Her3 and any optimization work for ErbB2 ligands will not predictably be applicable to Her1 or Her3 since they are different receptors with different biological properties and structures.

To this extent, what is needed are compositions that can bind to Her1 and Her3 with improved binding affinity. The invention described herein provides solutions for this need and provides additional benefits as well.

### BRIEF SUMMARY OF THE INVENTION

The invention provides for compositions comprising Her 1 and/or Her3 variants which have been optimized to improve binding to its cognate ligand. Accordingly, in one aspect, the invention provides for multimers comprising an extracellular domain (ECD) from Her3, which has been optimized to improve binding to its cognate ligand, linked to a Her1 ECD. In one embodiment, the optimization is a Y246A mutation. In another embodiment, the optimized Her3 additionally containing a lysine at position 132. In one embodiment, the Her3 variant has a K132E mutation. In another embodiment, the Her3 variant has lysine at position 132 with the Y246A variant. In another embodiment, the Her3 variant has lysine at position 132 without the Y246A variant. In another embodiment, the Her3 variant is truncated. In another embodiment, the truncated Her3 also has a lysine at position 132.

In another aspect, the invention provides for multimers comprising an extracellular domain (ECD) from Her1, which has been optimized to improve binding to its cognate ligand, linked to a Her3 ECD. In one embodiment, the Her1 ECD has a T15S mutation (or T39S if counting residues with the signal sequence peptide). In another embodiment, the Her1ECD has a T15S and G564S mutations.

The invention also provides for the compositions of Her 1 and Her3 variants which are associated with each other as homodimers. In one embodiment, a Her1 homodimer is formed with T15S and G564S mutations. In another embodiment a Her3 homodimer is formed with Y246A mutation. In another aspect, the invention provides for composition of Her3 variants which are associated with Her1 ECD as a heterodimer. In some embodiments, the Her1 ECD has also been optimized to improve binding to its cognate ligands (e.g., T15S or T15S/G564S mutations). The optimization is selected from the group consisting of: domain 4 deletion, T39S (or T15S without the signal sequence), S193N/E330D/G588S, and T39S/G564S.

The invention additionally provides for a composition comprising a mixture of Her1/Her1 homodimers, Her1/Her3 heterodimers and Her3/Her3 homodimers where the Her 1 and/or the Her3 component has been optimized to improve ligand binding. In some aspects, any of the multimers of homodimer or heterodimer are linked to the Fc receptor by using linker, such as an universal linker.

The invention also provides for pharmaceutical compositions and/or medicaments comprising optimized Her1 and/or optimized Her3 variants. The invention also provides for the use of optimized Her1 and/or optimized Her3 variants in the manufacture for a medicament for inhibiting cancer cell growth. In another embodiment, optimized Her1 and/or optimized Her3 variants is used n the manufacture for a medicament for treating abnormal growth of cells expressing Her1 and/or Her3.

The invention also provides for methods of using such compositions for inhibiting the growth of cancer cells. In some embodiments, the inhibition of cancer cell growth is in vivo used as a therapeutic composition. In other embodiments, the inhibition of cancer cell growth is in vitro. In yet other embodiments, the composition comprising optimized Her1 and/or optimized Her3 variants are used for ex vivo treatments.

### BRIEF DESCRIPTION OF THE FIGURES

FIG. 1 depicts the HER Family and its ligands.

FIG. 2 depicts a chart that summarizes the nomenclature for Hermodulins.

FIG. 3 depicts some Hermodulin molecules with uniform linker and Fc.

FIG. 4 depicts results from experiments for optimization of Her1 and Her3 ECD.

FIG. 5 shows results from experiments measuring the binding affinity for HFD300 and HFD300.1 to its ligand.

FIG. 6 depicts binding results for RB242.1B (“B” as part of the nomenclature indicates the use of the universal linker).

FIG. 7 depicts results from experiments testing for Hermodulin homodimers inhibition of Her3 phosphorylation.

FIG. 8 shows the results of experiments testing for relative inhibition of receptor phosphorylation by Hermodulin heterodimers.

FIG. 9 shows the results of experiments testing for relative inhibition of receptor phosphorylation where heterodimers are compared to homodimers when normalized for the number of ligand-binding sites.

FIG. 10 show the results of average fold improvement for various ECD pairings that show that the pairings may influence heterodimer activity

FIG. 11 depicts results from experiments testing for Hermodulins inhibition of NRG-induced MCF7 proliferation.

FIG. 12 shows the result of experiments testing for inhibition of NRG-induced T47D proliferation by Hermodulins

FIG. 13 shows that ligand binding affinities of RB200 were optimized via a high throughput rational mutagenesis process.

FIG. 14 shows that Hermodulin can inhibit ligand-induced cell proliferation.

FIG. 15 shows the pharmacokinetics of RB200 in rats. RB200 was administered as a single intravenous (IV) or intraperitoneal (IP) dose of 15 mg/kg in normal rats, plasma samples were collected at various time points. Plasma concentrations of RB200 were analyzed via Hermodulin-specific ELISA using anti-HER1 and anti-HER3 as capture antibodies, anti-human Fc-HRP as detection antibody. Data is mean±SEM of 2-3 rats per time point. Pharmacokinetic parameters were calculated using Sigma Plot 10.0.1.

FIG. 16 shows plasma concentrations of RB200 and RB242 in nude mice. RB200 and RB242 were administered as a single ip dose of 30 mg/kg in CD-1 nude mice, plasma samples were collected at 24 hr and day 7. Plasma concentrations of RB200 and RB242 were determined by Hermodulin-specific ELISA. Data are plotted mean plasma concentration (±SD) of 4 mice per time point.

FIG. 17 shows that the optimized bi-specific ligand trap RB242.1 is a designed triple mutant. RB242.1 demonstrats higher ligand binding affinity (Top Panels) and increased inhibitory activity in growth factor-induced HER phosphorylation (Middle panels) and tumor cell proliferation (bottom panels). KDs and EC50s are measured, and fold improvement over the parent/interim forms are indicated.

FIG. 18 shows high-affinity EGFR ligand binding is suppressed in the Fc-mediated EGFR/HER3 heterodimers. ¹²⁵I-ligand binding was performed in anti-Fc-coated 96-well plates with the indicated purified EGFR/HER3 heterodimers immobilized on the surface. Shown are ₁₂₅I-TGF-a binding (top), and ¹²⁵I-NRG1-β binding (bottom). Results are means±SEM of triplicate wells.

FIG. 19 shows that RB242 has restored high-affinity for EGFR ligands. Ligand binding was performed in anti-Fc-coated 96-well plates using the optimized ligand binding conditions as detailed in the Examples. Panels A and B show the saturation binding of Eu-EGF and Eu-NRG1-β. Panels C and D show the displacement of Eu-EGF with unlabeled TGF-a or HB-EGF. Results were representatives of three independent experiments and were normalized to fractions of receptors bound with ligands.

FIG. 20 shows in panel A that RB242 is more potent than RB200 in inhibition of proliferation of cultured tumor cells. The top panels show the results using serum-starved BxPC3 pancreatic cancer cells were treated with 3 nM of either TGF-a (top left) or NRG1-β (top right) for 3 days in the presence of increasing concentrations of RB200 or RB242. The bottom left panel shows results from serum-starved MCF7 cells that were treated with 3 nM of NRG1-β for 3 days in the presence of increasing concentrations of RB200 or RB242. The bottom right panel shows the proliferation of H1437 NSCLC cells in growth medium (RPMI1640/10% FBS) for 5 days in the presence of increasing concentrations of RB200 or RB242. Cell proliferation was quantified using standard techniques and discussed in the Examples. The results are means±SEM of 8 or 16 replicates. Approximate EC₅₀ values for BxPc3 cells were determined with the constraint type set to top constant equal to 100. Panel B shows that RB242 has improved anti-tumor activity in a mouse tumor xenograft model. Nude mice were transplanted with H1437 NSCLC cells subcutaneously as described in the Examples. When the tumor volume reached approximately 100 mm³, the mice were treated with either PBS vehicle (∘) or RB200 (▾) or RB242 (▴) at 12 mg per Kg administered intra-peritoneally 3 times weekly for 3 weeks. There were 9 mice per each treatment group. Data are expressed as mean tumor volume±SEM. **=P<0.01 by two way ANOVA with Bonferroni's post test.

### DETAILED DESCRIPTION

The invention provides for compositions comprising Her 1 and/or Her3 ligand binding domain which have been optimized for improved binding to its cognate ligand. These compositions are useful for inhibiting the activation of cells through capture of multiple HER ligands (growth factors). As used herein, these types of compositions can be pan-specific HER ligand traps (pan-HER) or also referred to herein as “Hermodulins.”

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety.

### General Description

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989. Cold Spring Harbor Press); Oligonucleotide Synthesis (M. J. Gait, ed., 1984); Animal Cell Culture (R. I. Freshney, ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991) and Short Protocols in Molecular Biology (Wiley and Sons, 1999).

### Definitions

As used herein, an extracellular domain (ECD) is the portion of the cell surface receptor that occurs on the surface of the receptor and includes the ligand binding site(s). For purposes herein, reference to an ECD includes any ECD-containing molecule, or portion thereof, so long as the ECD polypeptide does not contain any contigous sequence associated with another domain (i.e. transmembrane, protein kinase domain, or others) of a cognate receptor. Thus, for example, an ECD polypeptide includes alternative spliced isoforms of cell surface receptors (CSRs) where the isoform has an ECD-containing portion, but lacks any other domains of a cognate CSR, and also has additional sequences not associated or aligned with another domain sequence of a cognate CSR. These additional sequences can be intron-endoded sequences such as occur in intron fusion protein isoforms. Typically, the additional sequenes do not inhibit or interfere with the ligand binding and/or receptor dimerization activities of a CSR ECD polypeptide. An ECD polypeptide also includes hybrid ECDs.

As used herein, a multimerization domain refers to a sequence of amino acids that promotes stable interaction of a polypeptide molecule with another polypeptide molecule containing a complementary multimerization domain, which can be the same or a different multimerization domain to forms a stable multimer with the first domains. Generally, a polypeptide is joined directly or indirectly to the multimerization domain. Exemplary multimerization domains include the immunoglobulin sequences or portions thereof, leucine zippers, hydrophobic regions, hydrophilic regions, compatible protein-protein interaction domains such as, but not limited to an R subunit of PKA and an anchoring domain (AD), a free thiol that forms an intermolecular disulfide bond between two molecules, and a protuberance-into-cavity (i.e., knob into hole) and a compensatory cavity of identical or similar size that form stable multimers. The multimerization domain, for example, can be an immunoglobulin constant region. The immunoglobulin sequence can be an immunoglobulin constant domain, such as the Fc domain or portions thereof from IgG1, IgG2, IgG3 or IgG4 subtypes, IgA, IgE, IgD and IgM.

Compositions Comprising Optimized Her 1 and/or Her3

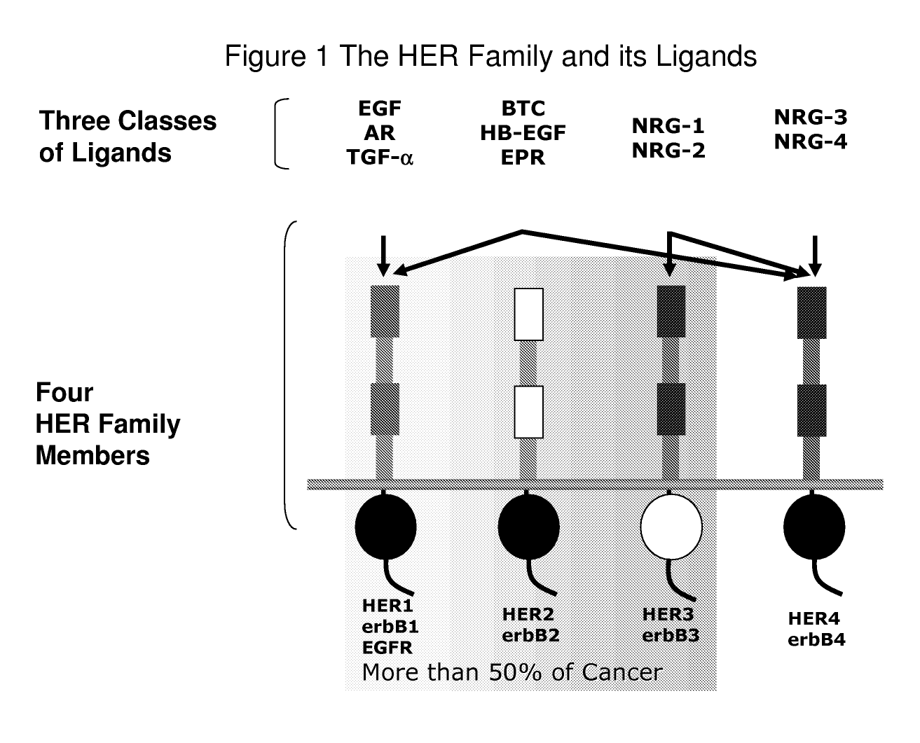

The invention provides for compositions comprising Her 1 and/or Her3 extracellular domain (ECD) which has been engineered (or optimized) for improved binding as compared to Her 1 and/or Her3 which have not been engineered. Such compositions have utility, for example, as a component for binding assays to its cognate ligand. In some aspects, the composition contains a multimer of Her3 ECD homodimer. The Her3 ECD can contain mutations, disclosed in greater detail below and in the Figures, which are useful for improving binding to its ligand. In other aspects, the composition contains a multimer of Her3 and Her1 heterodimer. For either one of Her1 or Her3 or both, optimization can be used to improve binding affinity to their ligands or additionally to improve other biological properties, including but not limited to, inhibiting phosphorylation of receptor tyrosine kinases, increasing bioavailability in an animal, better pharmacokinetics in vivo, inhibiting cell migration, reducing tumor volume, or halting tumor growth. In yet other aspects, the composition contains a mixture of Her1/Her1 homodimer (for example, a homodimer of HER1 with T15S, G564S mutation), Her1/Her3 heterodimer, and Her3/Her3 homodimer wherein the Her3 component has been engineered for improved binding to its ligands. FIG. 1 depicts the HER family and its ligands. The dysregulation of Her1, Her2 and Her3 account for over 50% of the current cases of cancer. The Examples detail the many variants that have been made and tested for optimal Her3 ligand binding.

It is to be understood that the invention also encompasses the combination of optimized Her3 and optimized Her1 to form a multimer (either homodimers or heterodimers) as well as mixtures of the Her1/Her1 homodimer, Her1/Her3 heterodimer, and Her3/Her3 homodimer.

### HER1 ECD Structure and Domain Organization

The extracellular portion of HER1 includes residues 1-621 of a mature HER1 receptor and contains subdomains I (amino acid residues 1-165), II (amino acid residues 166-313), III (amino acid residues 314-481), and IV (amino acid residues 482-621). The I, II, and III domains of HER1 have structural and sequence homology to the first three domains of the type I insulin-like growth factor receptor (IGF-1R, see e.g., Garret et al., (2002) Cell, 110:763-773). Similar to IGF-1R, the L domains (i.e. domains I and III) have a structure of a six turn β helix capped at each end by a helix and a disulfide bond. As compared to IGF-1R, the HER1 sequence includes amino acid insertions that contribute to biochemical structures important for mediating ligand binding by HER1. Among these include a V-shaped excursion (residues 8-18), which sits over the large β sheet of domain I to form a major part of the ligand binding interface. In domain III, a corresponding region forms a loop (residues 316-326) that also is involved in ligand binding. A third insert region present in domain III (residues 351-369) is an extra loop in the second turn of domain III. This loop is the epitope for various antibodies that prevent ligand binding (i.e., LA22, LA58, and LA90, see e.g., Wu et al., (1989) J Biol Chem., 264:17469-17475). In addition, other loops in the fourth turn of the B helix solenoid are involved in ligand binding.

TGF-α, a ligand for HER1, interacts with the large B sheets of both the L domains I and III of one receptor molecule. Similarly, the ligand EGF also interacts with both domains I and III of HER1, although the interaction of EGF with domain III is considered to be the major binding site for EGF (Kim et al., (2002) FEBS, 269: 2323-2329). Cross-linking studies have determined that the N- and C-terminal portions of the EGF ligand interact with domains I and III, respectively, of the HER1 receptor. Amino acid Gly441 in domain III, corresponding to mature full-length HER1, is involved in mediating binding to EGF via interaction with Arg45 of human EGF. A 40 kDa fragment of HER1 of 202 amino acids (corresponding to amino acids 302-503 of a mature HER1 polypeptide) is sufficient to retain full ligand-binding capacity of HER1 to EGF. This 202 amino acid portion contains all of domain III, and only a few residues each of domain II and domain IV (Kohda et al., (1993) JBC 268: 1976).

Domain II of EGFR contains eight disulfide-bonded modules. Domain II interacts with both domains I and III. The contacts with domain III occurs via modules 6 and 7, while modules 7 and 8 have a degree of flexibility thereby functioning to create a hinge in the ligand-free form of the EGFR molecule. A large ordered loop is formed from module 5 of domain II and projects directly away from the ligand binding site. This loop corresponds to residues 240-260 (also described as residues 242-259) and contains an antiparallel β-ribbon. The loop (also called the dimerization arm) is important in mediating intramolecuar interactions as well as mediating receptor-receptor contacts. In the inactive or “tethered” conformation of HER1, the loop contributes to intramolecular interactions by inserting between similar loop structures in modules 5 and 6 corresponding to amino acids 561-569 and 572-585, respectively, of a mature full-length ECD.

Deletion of the domain II loop abolishes the ability of the HER1 ECD to dimerize, thus showing its importance in facilitating intermolecular interactions. Dimerization is mediated by projection of the loop out across domain II of a second HER molecule in a space between domain I, II, and III. For example, contact is made by residues 244-253 of the dimerization arm with residues 229-239, 262-278, and 282-288 on the concave face of domain II in a second HER molecule. Tyr246 in domain II makes hydrogen bonds with Gly264 and Cys283 residues in a second HER molecule, and the phenyl rings of Tyr246 also interacts with Ser262 and Ser282 of an adjacent molecule. Other amino acid contacts between domain II of an EGFR and another HER molecule include Tyr251 with Phe263, Gly264, Tyr275, and Arg285; Pro248 with Phe230 and Ala265; Met253 with Thr278; and Tyr251 with Arg285. In addition, Asn247 and Asn256 are important for maintaining the loop in the appropriate conformation. Most all of these residues are conserved among HER family members and function similarly between HER family receptors. Further, proline residues occur in the loop in HER family receptors at any one of positions 243, 248, 255, and 257, with HER3 containing three prolines. The proline residues stabilize the conformation of the loop further. For example, HER1 contains prolines at position 248 and 257.

In addition to the involvement of domain IV (modules 5 and 6) in tethering of an inactive HER1 molecule, at least part of module 1 of domain IV of HER1 also appears to be required to maintain the structural integrity of an active HER1 molecule. For example, as mentioned above, a 40 kDa proteolytic fragment of HER1 containing all of domain III and part of domains II and IV retains full-ligand binding ability. The portion of domain IV present in this molecule corresponds to amino acids 482-503, including all of module 1. The amino acid corresponding to Trp492 in a mature HER1 molecule plays a role in maintaining stability of the HER1 molecule by interacting with a hydrophobic pocket in domain III. A recombinant molecule of HER1 containing all of domains I, II, and III but lacking all of domain IV is unable to bind ligand (corresponding to amino acids 1-476 of a mature HER1, see e.g., Elleman et al., (2001) Biochemistry 40:8930-8939). Thus, at least all or a portion of module 1 of domain IV appears to be required for the ligand binding ability of HER1. The remainder of domain IV is expendable for ligand binding and signaling. For example, normal ligand binding and signaling properties of HER1 are present in a HER1 molecule missing residues 521-603 of a mature HER1 polypeptide.

### HER3 ECD Structure and Domain Organization

The extracellular portion of HER3 includes residues 1-621 of a mature HER3 receptor and contains subdomains I (amino acid residues 1-166), II (amino acid residues 167-311), III (amino acid residues (312-480), and IV (amino acid residues 481-621). Like other HER family receptors, the structure of domains I, II, and III of HER3 can be superimposed with IGF-1R, and exhibit many of the same structural features as other HER receptors. For example, domains I and III of HER3 exhibit the a β-helical structure, interrupted by extended repeats of disulfide-containing modules. A high degree of interdomain flexibility exists between domains II and III, not exhibited by IGF-1R. In addition, HER3 exhibits the characteristic β-haripin loop or dimerization arm in domain II (corresponding to amino acids 242-259 of HER3). The β-hairpin loop provides for an intramolecular contact with conserved residues in domain IV resulting in a closed, or inactive HER3 structure. The residues important in this tethering interaction include interaction of Y246 with D562 and K583, F251 with G563, and Q252 with H565. Upon binding of ligand, a conformational change reorients domains I and III exposing the dimerization arm from the tethered structure to allow for receptor dimerization.

Unlike other HER family receptors, HER3 does not have a functional kinase domain. Alterations of four amino acid residues in the kinase region that are otherwise conserved among all protein tyrosine kinases render the HER3 kinase dysfunctional. HER3, however, retains tyrosine residues in its carboxy terminal domain and is capable of inducing cellular signaling upon appropriate activation and transphosphorylation. Thus, homodimers of HER3 cannot support linear signaling. The preferential dimerization partner for HER3 is HER2. As such, the invention provided herein is not to be expected in view of this dimerization preference. The ligands for Her3 include neuregulin-1 (NRG-1) and neuregulin-2 (NRG-2).

Components of ECD Multimers and the Formation of ECD Multimers

ECD heteromultimers include at least two different ECDs, or portions thereof for binding to ligand and/or dimerization. In exemplary embodiments herein, at least one of the component ECDs is a HER3 ECD. The ECDs in the heteromultimers or homomultimers are linked, whereby multimers, at least heterodimers or homodimers form. Any linkage is contemplated that permits or results in interaction of the ECDs to form a heteromultimer or homomultimer.

ECD Polypeptides

ECD polypepetides for use in the generation of ECD multimers provided herein can be all or part of an ECD of Her3 and/or Her1. As discussed in greater detail below, various methods can be used to generate variants of these ECD polypeptides that exhibit improved binding to its ligand(s). The ECD of Her3 and/or Her1 that is used can be full-length or a truncation and also encompasses the use of allelic variants.

Formation of ECD Multimers

ECD multimers, including HER ECD multimers, can be covalently-linked, non-covalently-linked, or chemically linked multimers of receptor ECDs, to form dimers, trimers, or higher multimers. In some instances, multimers can be formed by dimerization of two or more ECD polypeptides. Multimerization between two ECD polypeptides can be spontaneous, or can occur due to forced linkage of two or more polypeptides. In one example, multimers can be linked by disulfide bonds formed between cysteine residues on different ECD polypeptides. In another example, multimers can include an ECD polypeptide joined via covalent or non-covalent interactions to peptide moieties fused to the soluble polypeptide. Such peptides can be peptide linkers (spacers), or peptides that have the property of promoting multimerization. In an additional example, multimers can be formed between two polypeptides through chemical linkage, such as for example, by using heterobifunctional linkers.

Peptide Linkers

Peptide linkers can be used to produce polypeptide multimers, such as for example a multimer where one multimerization partner is all or a part of an ECD of a HER family receptor. In one example, peptide linkers can be fused to the C-terminal end of a first polypeptide and the N-terminal end of a second polypeptide. This structure can be repeated multiples times such that at least one, preferably 2, 3, 4, or more soluble polypeptides are linked to one another via peptide linkers at their respective termini. For example, a multimer polypeptide can have a sequence Z₁—X—Z₂, where Z₁ and Z₂ are each a sequence of all or part of an ECD of a cell surface polypeptide and where X is a sequence of a peptide linker. In some instances, Z₁ and/or Z₂ is a all or part of an ECD of a HER family receptor. In another example, Z₁ and Z₂ are the same or they are different. In another example, the polypeptide has a sequence of Z₁—X—Z₂(—X—Z)ₙ, where “n” is any integer, i.e. generally 1 or 2.

Typically, the peptide linker is of sufficient length to allow a soluble ECD polypeptide to form bonds with an adjacent soluble ECD polypeptide. Examples of peptide linkers include -Gly-Gly-, GGGGG, GGGGS or (GGGGS)ₙ, SSSSG or (SSSSG)ₙ, GKSSGSGSESKS, GGSTSGSGKSSEGKG, GSTSGSGKSSSEGSGSTKG, GSTSGSGKPGSGEGSTKG, EGKSSGSGSESKEF, or AlaAlaProAla or (AlaAlaProAla)ₙ, where n is 1 to 6, such as 1, 2, 3, or 4. In a preferred embodiment, the linker is GGGGG (also referred to herein as a “universal linker” and constructs with this linker have “B” designation at the end of its name).

Linking moieties are described, for example, in Huston et al. (1988) PNAS 85:5879-5883, Whitlow et al. (1993) Protein Engineering 6:989-995, and Newton et al., (1996) Biochemistry 35:545-553. Other suitable peptide linkers include any of those described in U.S. Pat. Nos. 4,751,180 or 4,935,233, which are hereby incorporated by reference. A polynucleotide encoding a desired peptide linker can be inserted between, and in the same reading frame as a polynucleotide encoding a soluble ECD polypeptide, using any suitable conventional technique. In one example, a fusion polypeptide has from two to four soluble ECD polypeptides, including one that is all or part of a HER ECD polypeptide, separated by peptide linkers.

Typically, the immunoglobulin portion of an ECD chimeric protein includes the heavy chain of an immunoglobulin polypeptide, most usually the constant domains of the heavy chain. In one example, an immunoglobulin polypeptide chimeric protein can include the Fc region of an immunoglobulin polypeptide. Typically, such a fusion retains at least a functionally active hinge, C_{H}2 and C_{H}3 domains of the constant region of an immunoglobulin heavy chain. Another exemplary Fc polypeptide is set forth in PCT application WO 93/10151, and is a single chain polypeptide extending from the N-terminal hinge region to the native C-terminus of the Fc region of a human IgG1 antibody. The precise site at which the linkage is made is not critical: particular sites are well known and can be selected in order to optimize the biological activity, secretion, or binding characteristics of the ECD polypeptide. For example, other exemplary Fc polypeptide sequences begin at amino acid C109 or P113 of the sequence (see e.g., US 2006/0024298).

In addition to hIgG1 Fc, other Fc regions also can be included in the ECD chimeric polypeptides. For example, where effector functions mediated by Fc/FcγR interactions are to be minimized, fusion with IgG isotypes that poorly recruit complement or effector cells, such as for example, the Fc of IgG2 or IgG4, is contemplated. Additionally, the Fc fusions can contain immunoglobulin sequences that are substantially encoded by immunoglobulin genes belonging to any of the antibody classes, including, but not limited to IgG (including human subclasses IgG1, IgG2, IgG3, or IgG4), IgA (including human subclasses IgA1 and IgA2), IgD, IgE, and IgM classes of antibodies. Further, linkers can be used to covalently link Fc to another polypeptide to generate an Fc chimera.

Modified Fc domains also are contemplated herein for use in chimeras with ECD polypeptides, see e.g. U.S. Patent Publication No. US 2006/0024298; and International Patent Publication No. WO 2005/063816 for exemplary modifications. In some examples, the Fc region is such that it has altered (i.e. more or less) effector function than the effector function of an Fc region of a wild-type immunoglobulin heavy chain. The Fc regions of an antibody interact with a number of Fc receptors, and ligands, imparting an array of important functional capabilities referred to as effector functions.

Thus, a modified Fc domain can have altered affinity, including but not limited to, increased or low or no affinity for the Fc receptor. For example, the different IgG subclasses have different affinities for the FcγRs, with IgG1 and IgG3 typically binding substantially better to the receptors than IgG2 and IgG4. In addition, different FcγRs mediate different effector functions. FcγR1, FcγRIIa/c, and FcγRIIIa are positive regulators of immune complex triggered activation, characterized by having an intracellular domain that has an immunoreceptor tyrosine-based activation motif (ITAM). FcγRIIb, however, has an immunoreceptor tyrosine-based inhibition motif (ITIM) and is therefore inhibitory. Thus, altering the affinity of an Fc region for a receptor can modulate the effector functions induced by the Fc domain.

In one example, an Fc region is used that is modified for optimized binding to certain FcγRs to better mediate effector functions, such as for example, ADCC. In another example, a variety of Fc mutants with substitutions to reduce or ablate binding with FcγRs also are known. Such muteins are useful in instances where there is a need for reduced or eliminated effector function mediated by Fc. This is often the case where antagonism, but not killing of the cells bearing a target antigen is desired. Exemplary of such an Fc is an Fc mutein described in U.S. Pat. No. 5,457,035. In some instances, an ECD polypeptide Fc chimeric protein provided herein can be modified to enhance binding to the complement protein C1q. In an additional example, an Fc region can be utilized that is modified in its binding to FcRn, thereby improving the pharmacokinetics of an ECD-Fc chimeric polypeptide. FcRn is the neonatal FcR, the binding of which recycles endocytosed antibody from the endosomes back to the bloodstream. This process, coupled with preclusion of kidney filtration due to the large size of the full length molecule, results in favorable antibody serum half-lives ranging from one to three weeks. Binding of Fc to FcRn also plays a role in antibody transport. Exemplary modifications in an Fc protein for enhanced binding to FcRn include modifications of amino acids corresponding to T34Q, T34E, M212L, and M212F.

Typically, a polypeptide multimer is a dimer of two chimeric proteins created by linking, directly or indirectly, two of the same or different ECD polypeptide to an Fc polypeptide. In some examples, a gene fusion encoding the ECD-Fc chimeric protein is inserted into an appropriate expression vector. The resulting ECD-Fc chimeric proteins can be expressed in host cells transformed with the recombinant expression vector, and allowed to assemble much like antibody molecules, where interchain disulfide bonds form between the Fc moieties to yield divalent ECD polypeptides. Typically, a host cell and expression system is a mammalian expression system can be used to allow for glycosylation of the appropriate amino acids.

The resulting chimeric polypeptides containing Fc moieties, and multimers formed therefrom, can be easily purified by affinity chromatography over Protein A or Protein G columns. Where two nucleic acids encoding different ECD chimeric polypeptides are transformed into cells, the formation of heterodimers must be biochemically achieved since ECD chimeric molecules carrying the Fc-domain will be expressed as disulfide-linked homodimers as well. Thus, homodimers can be reduced under conditions that favor the disruption of inter-chain disulfides, but do no effect intra-chain disulfides. Typically, chimeric monomers with different extracellular portions are mixed in equimolar amounts and oxidized to form a mixture of homo- and heterodimers. The components of this mixture are separated by chromatographic techniques. Alternatively, the formation of this type of heterodimer can be biased by genetically engineering and expressing ECD fusion molecules that contain an ECD polypeptide, followed by the Fc-domain of hIgG, followed by either c-jun or the c-fos leucine zippers. Since the leucine zippers form predominantly heterodimers, they can be used to drive the formation of the heterodimers when desired. ECD chimeric polypeptides containing Fc regions also can be engineered to include a tag with metal chelates or other epitope. The tagged domain can be used for rapid purification by metal-chelate chromatography, and/or by antibodies, to allow for detection of western blots, immunoprecipitation, or activity depletion/blocking in bioassays.

### Methods of Producing Optimized Her 1 and 3 ECDs

Any suitable method for generating the chimeric polypeptides between ECDs, portions thereof, particularly portions sufficient for ligand binding and/or receptor dimerization, and also alternatively splice portions, and a multimerization domain can be used. These methods are known to one of skill in the art. Similarly, formation of multimers from the chimeric polypeptides, can be achieved by any method known to those of skill in the art. As noted, the multimers typically include and ECD from at least one HER family member, typically a HER1 or a HER3.

ECD polypeptides also can be synthesized using automated synthetic polypeptide synthesis. Cloned and/or in silico-generated polypeptide sequences can be synthesized in fragments and then chemically linked. Alternatively, chimeric molecules can be synthesized as a single polypeptide. ECD-encoding nucleic acid molecules, including ECD fusion-encoding nucleic acid molecules, can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

As discussed further in the Examples section, members of the Her family, such as Her3, can be engineered to optimize its binding capabilities to its ligand. This can be accomplished by using a variety of methods known to one of skill in the art. A computer-aided program can be used to predict the likely areas for mutation. This can be followed by amino acid mutagenesis using standard molecular biology techniques and then ligand binding screening to identify the most optimized binders.

DNA encoding a chimeric polypeptide, such as any provided herein, is transfected into a host cell for expression. In some instances where ECD multimeric polypeptides are desired whereby multimerization is mediated by a multimerization domain, then the host cell is transformed with DNA encoding separate chimeric ECD molecules that will make the multimer, with the host cell optimally being selected to be capable of assembling the separate chains of the multimer in the desired fashion. Assembly of the separate monomer polypeptides is facilitated by interaction of each respective multimerization domain, which is the same or complementary between chimeric ECD polypeptides. Where HER family receptor ECDs, or portions thereof, are one or both ECD portions of the multimeric polypeptide, the multimerization domain is selected such that assembly of the monomers orients the dimerization arm of the HER molecule away from the partner multimer molecule. This orientation is referred to as “back-to-back” and ensures that the dimerization arm is accessible for dimerization with a cognate HER on the cell surface.

ECD polypeptides, including chimeric ECD polypeptides, can be expressed in any organism suitable to produce the required amounts and form of polypeptide needed for administration and treatment. Generally, any cell type that can be engineered to express heterologous DNA and has a secretory pathway is suitable. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

The generation of these ECD polypeptide multimers, including without limitation the optimization, multimerization, modifications, and linkages, may also be performed according to the methods disclosed in WO 2007/146959, which is specifically incorporated by reference in its entirety.

### Purification

ECD polypeptides and chimeric ECD polypeptides, including ECD polypeptide multimers, can be isolated using various techniques well-known in the art. One skilled in the art can readily follow known methods for isolating polypeptides and proteins in order to obtain one of the isolated polypeptides or proteins provided herein. These include, but are not limited to, immunochromatography, HPLC, size-exclusion chromatography, and ion-exchange chromatography. Examples of ion-exchange chromatography include anion and cation exchange and include the use of DEAE Sepharose, DEAE Sephadex, CM Sepharose, SP Sepharose, or any other similar column known to one of skill in the art. In some preferred embodiments, the protein purification is accomplished by using Protein A, Ni-Sepharose, Nickel His Trap or Anti-EGFR Affibody Sepharose.

Isolation of an ECD polypeptide or ECD multimer polypeptide from the cell culture media or from a lysed cell can be facilitated using antibodies directed against either an epitope tag in a chimeric ECD polypeptide or against the ECD polypeptide and then isolated via immunoprecipiation methods and separation via SDS-polyacrylamide gel electrophoresis (PAGE). Alternatively, an ECD polypeptide or chimeric ECD polypeptide including ECD multimers can be isolated via binding of a polypeptide-specific antibody to an ECD polypeptide and/or subsequent binding of the antibody to protein-A or protein-G sepharose columns, and elution of the protein from the column. The purification of an ECD polypeptide also can include an affinity column or bead immobilized with agents which will bind to the protein, followed by one or more column steps for elution of the protein from the binding agent. Examples of affinity agents include concanavalin A-agarose, heparin-toyopearl, or Cibacrom blue 3Ga Sepharose. A protein can also be purified by hydrophobic interaction chromatography using such resins as phenyl ether, butyl ether, or propyl ether. More than one column can be used to achieve greater purity.

### Assays to Assess or Monitor ECD Multimer Activities

Generally, an ECD multimer modulates one or more biological activities of one or more, typically two or more, cognate cell surface receptor (CSR) or other interacting CSR. In vitro and in vivo assays can be used to monitor a biological activity of an ECD multimer. Exemplary in vitro and in vivo assays are provided herein to assess the biological activity of HER ECD multimers. Assays to test for the effect of ECD multimers on RTK activity include, but are not limited to, kinase assays, homodimerization and heterodimerization assays, protein:protein interaction assays, structural assays, cell signaling assays and in vivo phenotyping assays. Assays also include the use of animal models, including disease models in which a biological activity can be observed and/or measured. Dose response curves of an ECD multimer in such assays can be used to assess modulation of biological activities and as well as to determine therapeutically effective amounts of an ECD multimer for administration. Exemplary assays are described below.

1. Kinase/Phosphorylation Assays

Kinase activity can be detected and/or measured directly and indirectly. For example, antibodies against phosphotyrosine can be used to detect phosphorylation of an RTK. For example, activation of tyrosine kinase activity of an RTK can be measured in the presence of a ligand for an RTK. Transphosphorylation can be detected by anti-phosphotyrosine antibodies. Transphosphorylation can be measured and/or detected in the presence and absence of an ECD multimer, thus measuring the ability of an ECD multimer to modulate the transphosphorylation of an RTK. Briefly, cells expressing an RTK can be exposed to an ECD multimer and treated with ligand. Cells are lysed and protein extracts (whole cell extracts or fractionated extracts) are loaded onto a polyacrylamide gel, separated by electrophoresis and transferred to membrane, such as used for western blotting. Immunoprecipitation with anti-RTK antibodies also can be used to fractionate and isolate RTK proteins before performing gel electrophoresis and western blotting. The membranes can be probed with anti-phosphotyrosine antibodies to detect phosphorylation as well as probed with anti-RTK antibodies to detect total RTK protein. Control cells, such as cells not expressing RTK isoform and cells not exposed to ligand can be subjected to the same procedures for comparison.

Tyrosine phosphorylation also can be measured directly, such as by mass spectroscopy. For example, the effect of an ECD multimer on the phosphorylation state of an RTK can be measured, such as by treating intact cells with various concentrations of an ECD multimer and measuring the effect on activation of an RTK. The RTK can be isolated by immunoprecipitation and trypsinized to produce peptide fragments for analysis by mass spectroscopy. Peptide mass spectroscopy is a well-established method for quantitatively determining the extent of tyrosine phosphorylation for proteins; phosphorylation of tyrosine increases the mass of the peptide ion containing the phosphotyrosine, and this peptide is readily separated from the non-phosphorylated peptide by mass spectroscopy.

2. Complexation/Dimerization

Complexation, such as dimerization of RTKs and ECD multimers can be detected and/or measured. For example, isolated polypeptides can be mixed together, subject to gel electrophoresis and western blotting. RTKs and/or ECD multimers also can be added to cells and cell extracts, such as whole cell or fractionated extracts, and can be subject to gel electrophoresis and western blotting. Antibodies recognizing the polypeptides can be used to detect the presence of monomers, dimers and other complexed forms. Alternatively, labeled RTKs and/or labeled ECD multimers can be detected in the assays. Such assays can be used to compare homodimerization of an RTK or heterodimerization of two or more RTKs in the presence and absence of an ECD multimer. Assays also can be performed to assess the ability of an ECD multimer to dimerize with an RTK. For example a HER3 ECD multimer can be assessed for its ability to heterodimerize with HER1.

3. Ligand Binding

Generally, RTKs bind one or more ligands. As discussed above, FIG. 1 illustrates some ligands that bind to members of the HER family. Ligand binding modulates the activity of the receptor and thus modulates, for example, signaling within a signal transduction pathway. Ligand binding to an ECD multimer and ligand binding of an RTK in the presence of an ECD multimer can be measured. For example, labeled ligand such as radiolabeled ligand can be added to purified or partially purified RTK in the presence and absence (control) of an ECD multimer. Immunoprecipitation and measurement of radioactivity can be used to quantify the amount of ligand bound to an RTK in the presence and absence of an ECD multimer. An ECD multimer also can be assessed for ligand binding such as by incubating an ECD multimer with labeled ligand and determining the amount of labeled ligand bound by an ECD multimer, for example, as compared to an amount bound by a wildtype or predominant form of a corresponding RTK. The Examples also lists other ways of detecting ligand binding.

4. Cell Proliferation Assays

HER family receptors are involved in cell proliferation. Effects of an ECD multimer on cell proliferation can be measured. Cells to be tested typically express the target RTK receptor. For example, ligand can be added to cells expressing an RTK. An ECD multimer can be added to such cells before, concurrently or after ligand addition and effects on cell proliferation measured. The level of proliferation of the cells can be assessed by labeling the cells with a dye such as Alamar Blue or Crystal Violet, or other similar dyes, followed by an optimal density measurement. MTT [3-(4,5-dimethylthiazol-2-yl)-2,5-diphenyltetrazolium bromide] also can be used to assess cell proliferation. The use of MTT as a proliferation reagent is based on the ability of a mitochondrial dehydrogenase enzyme from viable cells to cleave the tetrzolium rings of the pale yellow MTT and form dark blue formazan crystals which accumulates in healthy cells as it is impermeable to cell membranes. Solubilization of cells by the addition of a detergent results in the release and solubilization of the crystals. The color, which is directly proportional to the number of viable, proliferating cells, can be quantified by spectrophotometric means. Thus, after incubation of selected cells with an ECD multimer in the presence or absence of ligand, MTT can be added to the cells, the cells can be solublized with detergent, and the absorbance read at 570 nm. Alternatively, cells can be pre-labeled with a radioactive label such as 3H-tritium, or other fluorescent label such as CFSE prior to proliferation experiments.

5. Cell Disease Model Assays

Cells from a disease or condition or which can be modulated to mimic a disease or condition can be used to measure/and or detect the effect of an optimized Her3 multimer. An optimized Her3 multimer is added or expressed in cells and a phenotype is measured or detected in comparison to cells not exposed to or not expressing an ECD multimer. Such assays can be used to measure effects including effects on cell proliferation, metastasis, inflammation, angiogenesis, pathogen infection and bone resorption.

6. Animal Models

Animal models can be used to assess the effect of an optimized Her1 and/or Her3 multimers. For example, the effects of an ECD multimer on cancer cell proliferation, migration and invasiveness can be measured in an animal model of cancer. In one such assay, cancer cells such as ovarian cancer cells, after culturing in vitro, are trypsinized, suspended in a suitable buffer and injected into mice (e.g., into flanks and shoulders of model mice such as Balb/c nude mice). Mice are co-administered either before, concurrently, or after the administration of cancer cells to the mice by any suitable route of administration (i.e. subcutaneous, intravenous, intraperitoneal, and other routes). Tumor growth is monitored over time. Similar assays can be performed with other cell types and animal models, for example, murine lung carcinoma (LLC) cells and C57BL/6 mice and SCID mice. Tumor growth can be compared to mice not administered with an ECD multimer, or to mice who are deficient in the respective cognate receptor or interacting receptor of the ECD multimer.

### Methods of Use

The compositions disclosed herein have various uses. In one aspect, the Hermodulins can be used to inhibit the growth of cancerous cells. As shown in Examples and Figures, Hermodulins of this invention inhibit the proliferation of cancerous cells that have been induced by natural Her 1 and/or Her3 ligands and to an extent that would be unexpected to one of ordinary skill in the art. Hermodulins comprising optimized Her1 and/or Her1 can be administered in an effective amount to an individual in need thereof, for example, in an individual with cancer. The cancer can be any type of cancer which would benefit the individual being treated. Examples of cancer to be treated herein include, but are not limited to, carcinoma, lymphoma, blastoma, sarcoma, and leukemia or lymphoid malignancies. Additional examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, renal cell cancer, esophageal cancer, glioma, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Another example of an individual in need thereof is an individual who suffers from abnormal growth of cells expressing Her1 and/or Her3. The Hermodulins can also be used to reduce tumor volume and/or to inhibit the growth of a tumor. Tumor can encompass multiple types of tumors, including but not limited to, cancerous tumors, blood-based tumors and solid tumors. The Hermodulins of the invention can be used to treat and/or ameliorate other conditions, including those involving cell proliferation and/or migration, including those involving pathological inflammatory responses, non-malignant hyperproliferative diseases, such as ocular conditions, skin conditions, conditions resulting from smooth muscle cell proliferation and/or migration, such as stenoses, including restenosis, atheroscelerosis, muscle thickening of the bladder, heart or other muscles, endometriosis, or rheumatoid arthritis.

Other diseases that can be treated with a Hermodulin provided herein include any disease or disorder mediated by a HER family receptor or its ligands including, but not limited to, aggressiveness, growth retardation, schizophrenia, shock, Parkinson's disease, Alzheimer's disease, cardiomyopathy congestive, preeclampsia, nervous system disease, and heart failure. It will be apparent to one of skill in the art that other uses are available based on the functional and biological effects that the compositions of Hermodulin have. The compositions disclosed herein can be used in combination with other agents. Combination therapies can be used with Hermodulins including anti-hormonal compounds, cardioprotectants, anti-cancer agents such as chemo therapeutics and growth inhibitory agents, and any other such as is described herein.

The Hermodulin can be formulated as a pharmaceutically acceptable composition. The compositions can be administered in a manner suitable for effecting biological effects. This can be by any suitable route of administration (i.e. subcutaneous, intravenous, intraperitoneal, oral, intradermal, and other routes). In other cases, the pharmaceutical compositions also can be formulated for local, topical or systemic administration. In some embodiments, the pharmaceutical composition is formulated for single dosage administration. In other embodiments, kits comprising a composition of optimized Hermodulins are contemplated within the scope of the invention. In some embodiments, the kits are optionally packaged with instructions. The kit can contain a single dose of Hermodulin or multiple doses. The Hermodulin may be one or more of the following: homodimer of optimized Her1/Her1 or optimized Her3/Her3, optimized heterodimer of Her1/Her3, or a mixture of the homodimers and heterodimer.

### Methods for Identifying, Screening and Making Additional Hermodulins

In addition to ECD multimers provided herein, other candidate Hermodulins can be identified. Provided herein are methods to identify Hermodulins, and screening assays therefor. The methods are designed to identify molecules that target ECD subdomains to interfere with ligand binding and/or receptor dimerization and/or tethering by identifying molecules, such as small molecules and polypeptides, that interact with regions on more than one HER receptor family member that are involved in these activities. Such therapeutics can simultaneously target several members of the HER family who do not have multiple coexpression of HER receptors.

One method that can be used for identifying pharmacologically active pan-HER therapeutic molecules is to use computer-aided optimization techniques to sort through the possible mutations that result in higher affinity binding to the ligand(s). The Examples provide guidance on how such computer-aided optimization techniques can be used and provide working examples of optimized Her3 generated with the use of computer-aided optimization. For examples, HER1, HER2, HER3 or HER4 with enhanced binding to ligands may be generated this way and used as components to make heteromultimers, homomultimers and mixtures of both.

Hermodulins identified in the methods described above can be tested for their ability to functionally modulate one or more HER activity. Such activities are known to those of skill in the art and are described herein. Exemplary of such assays include ligand binding, cell proliferation, cell phosphorylation, and complexation/dimerization. Thus, any candidate identified herein as a candidate based on high affinity binding to a HER molecule or portion thereof, can be tested in further screening assays to determine if the candidate therapeutic possesses pan-HER therapeutic properties, i.e. inhibitory properties against HER activation.

### Examples

The following examples are included for illustrative purposes only and are not intended to limit the scope of the invention.

### Example 1

### Engineering Her3 with Improved Binding

The Her family and its ligands are depicted in FIG. 1. Computer modeling of HER1 ligand binding domain was performed using the co-crystal structures of EGFR-EGF (PDB code IMOX-chain C; Ogiso H et al. Cell (2002) 775-787) and EGFR-TGFa (PDB code 1IVO-chain C, Garrett, T. P. J 2002).). The Her3 portion of the ligand trap was improved for binding by using a combination of computational redesign, single amino acid mutagenesis, high throughput ligand binding screening, and then selection for the best optimized binders. For further experimentation, the expression was scaled-up and subject to some purification steps.

Computational Design

Computer modeling of HER3 ligand binding domain was done using the structure information of HER3 ECD (PDB code IM6B, Cho 2002, Schwede T, Kopp J, Guex N, and Peitsch M C (2003). The designed optimization of ligand-receptor interaction was based on the physio-chemical proterties and classification of amino acids such as charged, polar, aromatic, etc. Also considered were residue volume, surface area, solvent accessibilities, etc. PAM250 matrix was used to aid for the prediction of amino acid substitution (W. A Pearson, Rapid and Sensitive Sequence Comparison with FASTP and FASTA, in Methods in Enzymology, ed. R. Doolittle (ISBN 0-12-182084-X, Academic Press, San Diego) 183(1990)63-98; and also M. O. Dayhoff, ed., 1978, Atlas of Protein Sequence and Structure, Vol. 5).

### Example 2

### High-Throughput Mutagenesis

Site-directed mutagenesis was performed by overlapping PCR which included three sequential PCR reactions each catalyzed by the thermo-stable DNA polymerase elongase supplemented with proof-reading DNA polymerase pfu (Invitrogen). HER1:Fc and HER3:Fc cDNAs were used as the PCR templates. Conditions set up for the first round PCR with 2 pairs of primers was 94° C. 2 min, 94° C. 45 sec, 60° C. 45 sec, 68° C. 3 min for 26 cycles. The two overlapping PCR fragments generated by the first round PCR were gel-purified, combined at 1 to 1 molar ratio, and used for the second round PCR. The second round PCR annealed the two overlapping PCR fragment using the condition of 94° C. 2 min, 94° C. 45 sec, 57° C. 45 sec, 68° C. 30 min for 8 cycles. In the third round PCR, the product of the second round PCR was used as the template. PCR amplification was conducted in the presence of a forward primer that covered the start codon and a reverse primer that covered the stop codon. The PCR condition was 94° C. 2 min, 94° C. 45 sec, 60° C. 45 sec, 68° C. 3 min for 26 cycles. PCR products bearing mutations were cloned into the Gateway System plasmid pDONR221 (Invitrogen). Designed mutations were confirmed by complete sequencing. Inserts in pDONR221 were then transferred to the expression vector pcDNA3.2-DEST (Gateway System, Invitrogen) by LR reaction following the manufacturer's instruction.

### Example 3

### Protein Expression and Purification

For ligand binding screening, sequence-confirmed HER1:Fc and HER3:Fc mutants were transiently transfected into HEK293T cells (ATCC) using Lipofectamine 2000 (Invitrogen). For expression of the Fc-mediated HER1/3 heterodimers, the HER1:Fc and HER3:Fc or their mutants were cotransfected into HEK293T cells. The serum-free condition media were collected 72 hrs after transfection. Levels of HER1:Fc and Her3:Fc homodimers was quantified using the human HER1 or HER3 ELISA detection Kit following the manufacturer's instruction (R&D Systems). To quantify the Fc-mediated heterodimers, the anti-HER3-coated ELISA plates were used for capture and the HER1 antibody was used for detection.

For scale-up expression of HER1/3 heterodimers, log phase CHO-S cells (Invitrogen) maintained in Pro-CHO5 (Lonza, Allendale, N.J.) were transferred into Wave Bio-Reactor (GE HealthCare) at 1×10⁶/mL in Pro-CHO5 (Lonza) supplemented with 8 mM of L-glutamine and 1× HT (Gibco). Next day, cells were co-transfected with the corresponding HER1:Fc and HER3:Fc cDNA constructs. The transfection was achieved by using the 25 Kdal linear PEI (Polysciences) at 12 mg/L. The volume of ProCHO5 was doubled 4 hrs after transfection. Transfected cell were maintained in Wave Bio-Rector for 7 days before the conditioned medium was harvested.

The Fc-mediated HER1/3 heterodimers were purified by using the following protocol: conditioned medium from co-transfected CHO-S cells (Invitrogen) was clarified, 10-fold concentrated, and applied to a MabSelect SuRe affinity column (GE Healthcare Biosciences AB, Sweden). Column was washed extensively with phosphate-buffered saline (PBS) containing 0.1% (v/v) TX-114 and eluted with an IgG elution buffer (Pierce, Rockville, Ill.). The eluted fractions were immediately neutralized with 1M Tris-HCL to pH 8.0. Pool of the protein-containing fractions was loaded onto a Ni-Sepharose column (GE-Healthcare Biosciences AB, Sweden). Column was washed with the Ni-Sepharose Buffer containing 25 mM of imidazole. Bound proteins were eluted with a 25-135 mM of gradient imidazole in the same buffer. The main heterodimer peak was typically eluted between 80-125 nM of imidazole. Pool of the heterodimer-containing fractions from the Ni-Sepharose column was exhaustively dialyzed at 4′C in PBS. Purity of the heterodimer preparations was determination by analytical reversed-phase HPLC

### Example 4

### Screening for Improved Ligand Binding

Screening for binding of Europium-labeled EGF (Eu-EGF) and NRG1β (Eu-NRG1β) by Delfia (PerkinElmer) was carried out in 96-well yellow plated (Perkin Elmer). Wells were coated with 100 μl of anti-human Fc antibody (5 ug/mL, Sigma-Aldrich) at room temperature (RT) overnight. Coated plates were rinsed 3 times with PBS/0.05% Tween-20 (wash buffer, WB) and blocked with PBS/1% BSA at RT for 2 hrs. Plates were again rinsed 3 times with WB. The Fc-fusion proteins in conditioned media from the transfected HEK293T cells were diluted with Delfia binding buffer to a concentration of 20 ng/well and were added to each well (100 μl/well). Plates were incubated at RT for 2 hrs and then rinsed 3 times with DELFIA wash buffer. The plates were then incubated with 100 μl of Eu-EGF (Perkin Elmer) or Eu-NRG1β (custom-labeled by PerkinElmer) at a concentration of 0.5 nM. The plates were incubated at RT for 2 hrs followed by three quick rinse with ice-cold Delfia wash buffer containing0.02% Tween-20. To quantify bound Eu-ligands 130 μl/well of Delfia enhancement solution was added and the plates were read on a fluorescence plate reader (Envision, model 2100, PerkinElmer).

Screening for TGFa and HB-EGF binding was carried out using the TGFa and HB-EGF ELISA Kit (R&D System). 96-well plates were coated with 100 μl of anti-human Fc antibody at 1 ug/mL at RT overnight. Plates were rinsed and blocked as described above. The Fc-fusion proteins in conditioned media were diluted with PBS/1% BSA to 20 ng/well and were added to wells at 100 μl/well. Plates were incubated at RT for 2 hrs, followed by 3 rinses with WB. TGFα and HB-EGF (R&D Systems) were diluted to 5 nM with PBS/1% BSA and were added to the plates. The plates were incubated at RT for 2 hrs followed by 3 rapidly rinse with ice cold WB. Bound ligands were detected using the biotinylated detection antibody against TGFα or HB-EGF. Subsequent ELISA color development steps follow the manufacturer's instruction.

Procedures for screening for HER1 ligand binding (Eu-EGF, TGFα, and HB-EGF) to the immobilized HER1/3 heterodimers using the conditioned media were identical to the screening for Eu-EGF, TGFα, and HB-EGF binding described above, except that the plates were pre-coated with anti-human HER3 antibody (DYC1769) at a concentration of 2 μg/mL and that 100 ng/well of Fc-fusion proteins from the conditioned media were used for ligand binding.

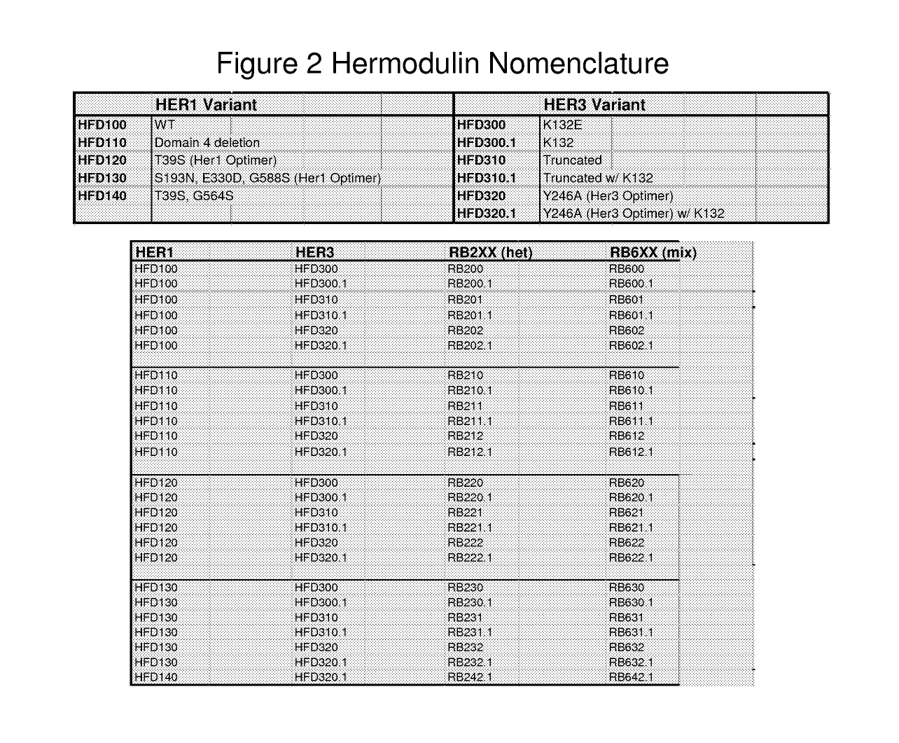

A variant with substitution at position 246 from tyrosine to alanine (Y246A) was predicted by modeling studies to give rise to high affinity and was screened and found to bind NRG1β. Previous work had optimized Her1 ECD to generate a variant called T39S (or without the 24 residue signal sequence, would be T155) called HFD120. The nomenclature of the various variants which have been constructed are depicted in FIG. 2 and below.

HER

| HER3 | HFD100 | HFD120 |
|---|---|---|
| HFD30 | RB20 | RB220 |
| HFD300.1 | RB200.1 | — |
| HFD320.1 | RB202.1 | RB222.1 |

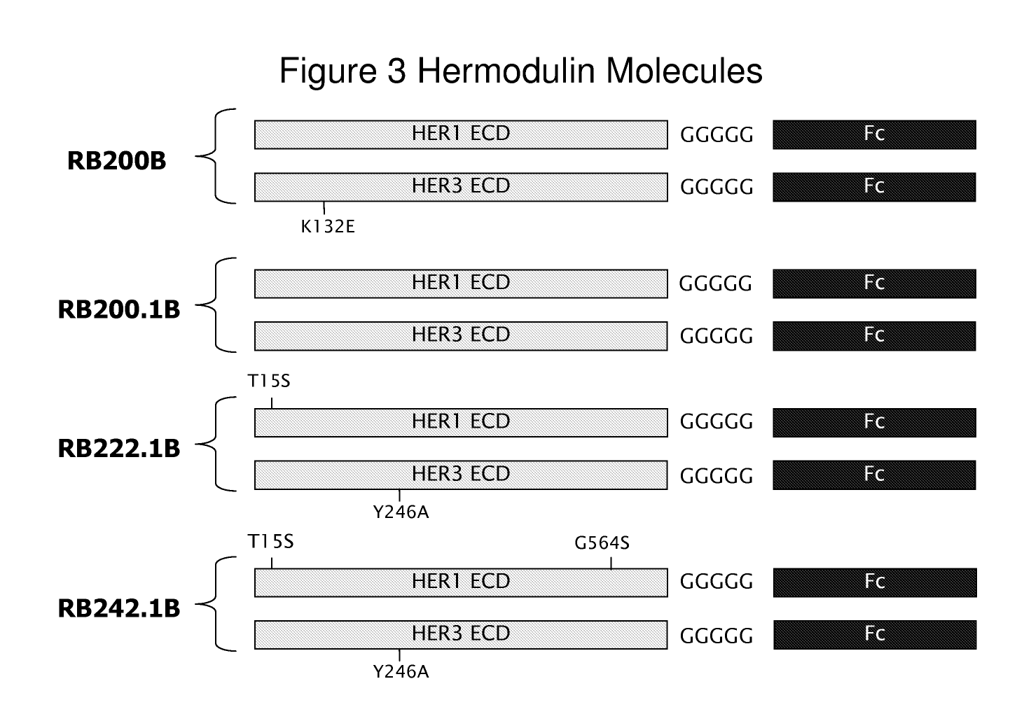

This nomenclature is used throughout this specification. These various Hermodulins with optimized Her1 and/or Her3 were linked to a uniform linker and Fc as shown in FIG. 3.

### Example 5

### Ligand Binding Assays

The various HFD constructs were screened using standard ligand binding assays including, but not limited to, I¹²⁵ labeling of ligand, DELFIA (Europium-labeled ligand), surface plasmon resonance (Biacore) and isothermal calorimetry. Exemplary protocols for saturation binding are as follows:

Eu-Ligand Saturation Binding and Displacement

Eu-EGF and Eu-NRG1β saturation binding and Eu-EGF displacement were identical to the EU-EGF binding screening described above, except that purified heterodimers were used and the heterodimer concentrations used for ligand binding were at least 10-fold lower than the KDs for the assayed ligands (CELL SURFACE RECEPTORS: A SHORT COURSE ON THEORY AND METHODS, Lee E. Limbird, 2004). For saturation binding with Eu-EGF, 30 ng/well of RB200 or 2 ng/well of RB242 were immobilized onto the anti-human Fc coated pates. For saturation binding with Eu-NRG1β, 2 ng/well of RB200 or RB242 were immobilized. Displacement assays were performed with Eu-EGF (at a concentration of 50 nM for RB200 or 5 nM for RB242) added to wells in the presence of increasing concentrations of the indicated unlabeled competitors.

125-Ligand Saturation Binding

¹²⁵I-EGF was purchased from GE-Healthcare. TGFα and HB-EGF (R&D Systems) were custom-labeled by GE-Healthcare. 96-well assay plates were coated with 5 μg/mL anti-human Fc antibody. Coated plates were washed and blocked as described above. Conditioned media or purified proteins diluted to 20 ng/well were immobilized in the anti-human Fe coated wells. Increasing concentrations of the ¹²⁵I-ligands were used to reach saturation binding. After binding, washed wells with bound ¹²⁵I-ligands were covered with 100 μl/well of a scintillation cocktail OptiPhase ‘SuperMix’ (PerkinElmer, Waltham, Mass.) and were read by Microbeta Trilux (PerkinElmer).

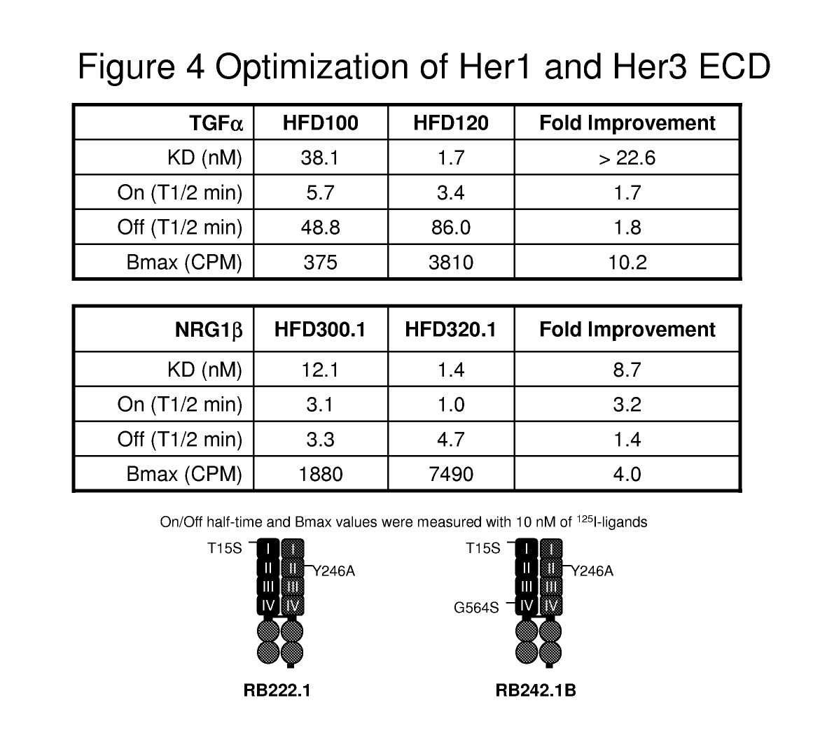

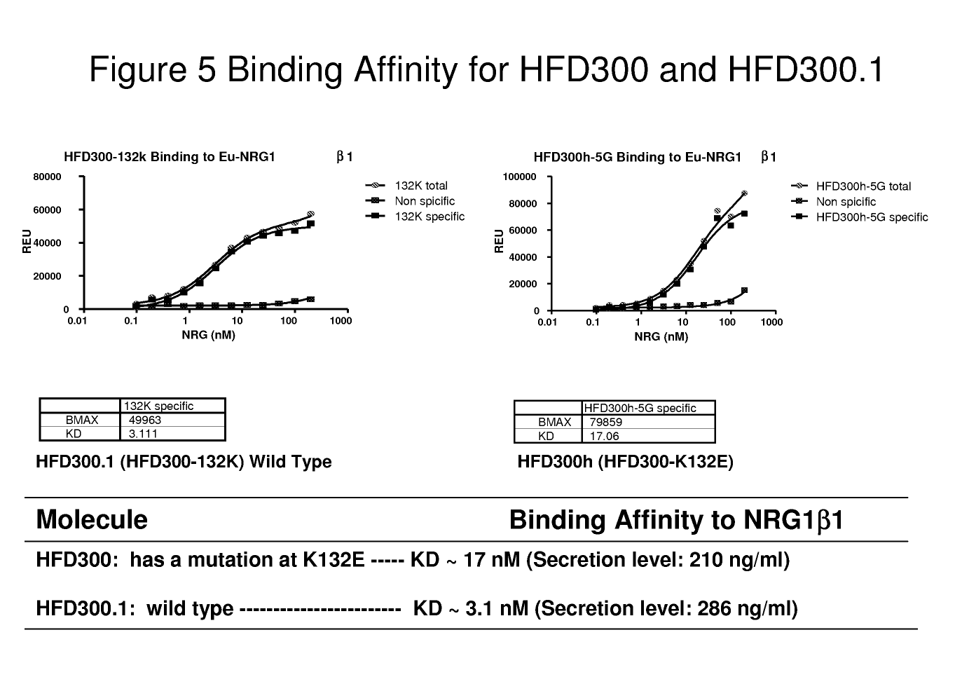

Her3 optimization was determined by binding to NRG1β1 (also referred to herein as NRGβ1). FIG. 4 shows data measuring on-off rates for the optimized Her1 and Her3 molecules. FIG. 5 shows the binding affinity for HFD300 and HFD300.1.

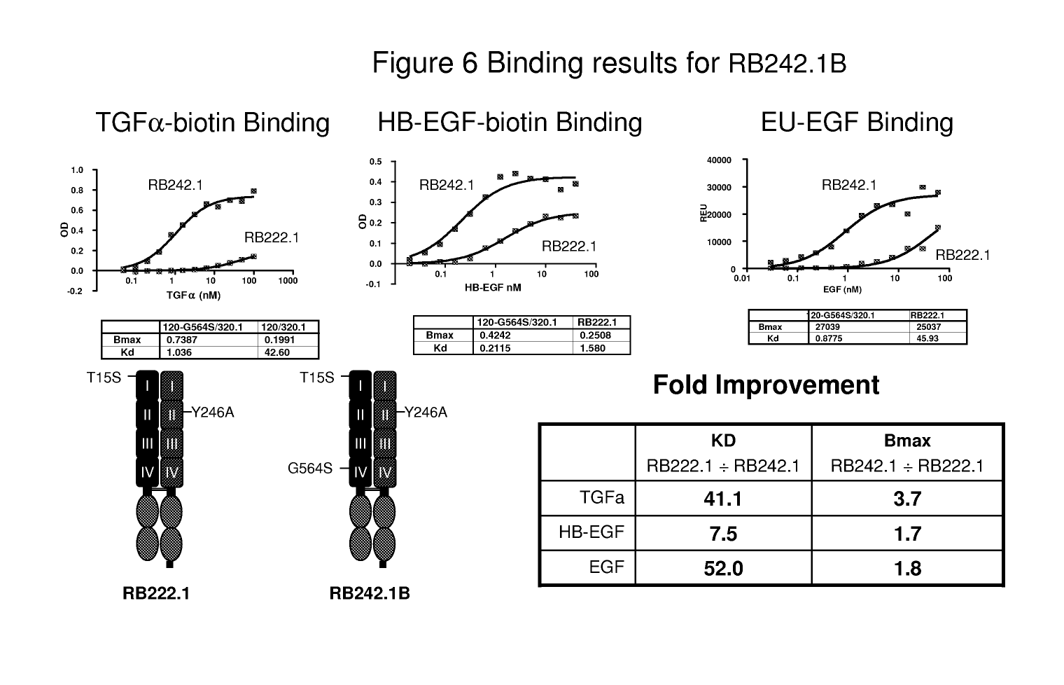

When RB242.1B was tested for binding affinity, the results (FIG. 6) showed that it overcame antagonism between HER1 and HER3 in ligand binding. The improvement was more than 30 fold over RB222.1 in its affinity for HER1 ligands.

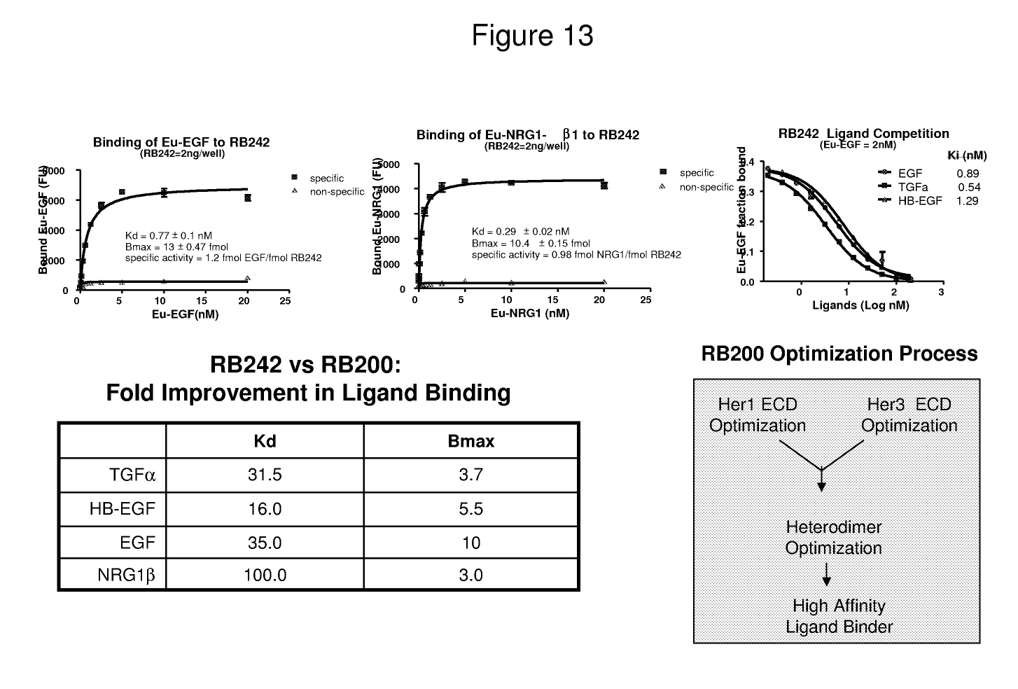

FIG. 13 shows that ligand binding affinities of RB200 were optimized via a high throughput rational mutagenesis process. An optimized hermodulin variant RB242 with sub-nanomolar affinities for both HER1 and HER3 ligands was identified. Binding of RB242 to other HER ligands such as TGF-α and HBEGF was assessed by competitive binding against Eu-EGF. The comparison of RB242 vs. RB200 in binding to different ligands is expressed as fold improvement in Kd and Bmax based on multiple determinations.

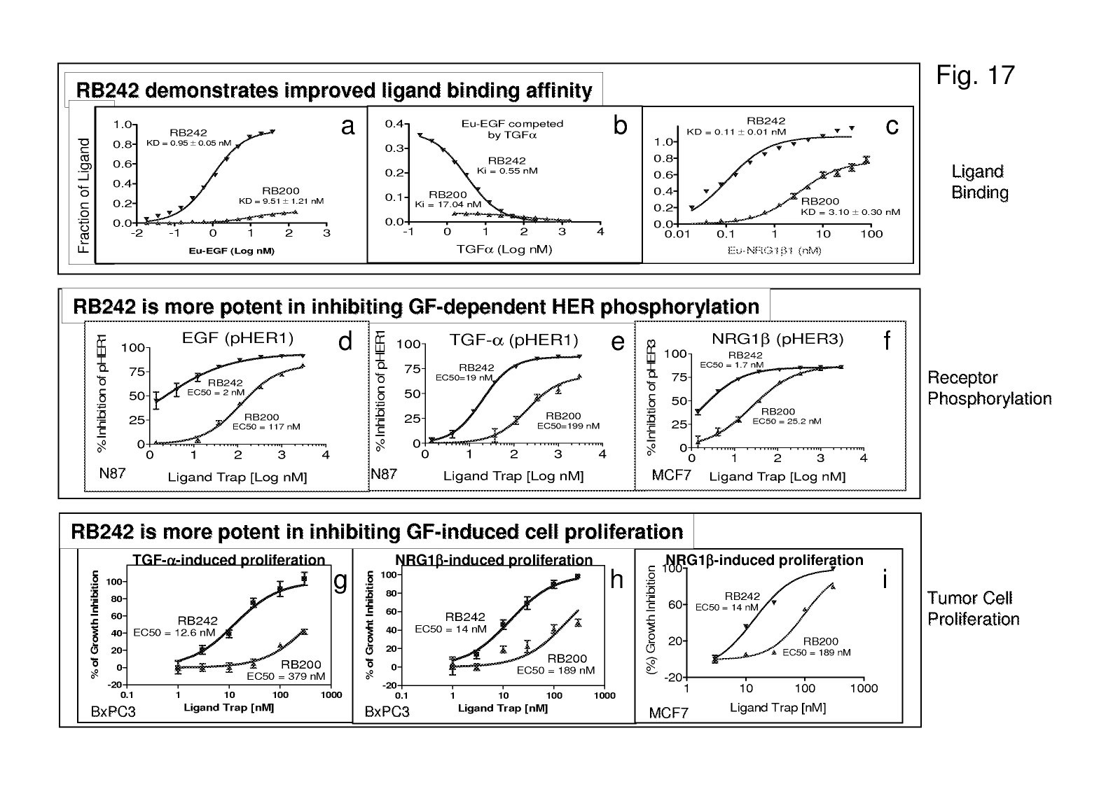

FIG. 17 (top panel) also shows that RB242 has improved ligand binding affinity.

### Example 6

### Inhibition of Phophorylation of RTK with Hermodulin Homodimers

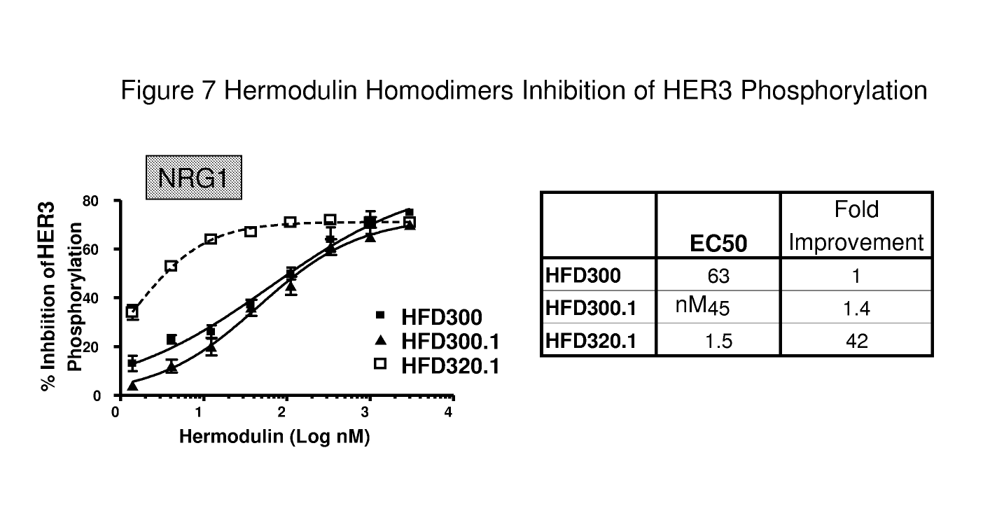

The optimized Her3 constructs were tested to see if they could inhibit NRG-stimulated phosphorylation of Her3. FIG. 7 depicts results from experiments testing for Hermodulin homodimers inhibition of HER3 phosphorylation. As shown, HFD320.1 showed an unexpected 42-fold improvement over HFD300.

### Example 7

### Inhibition of Phosphorylation by Hermodulin Heterodimers

Various constructs of Hermodulin heteromodulins were tested for determine their effect on phosphorylation. Phosphotyrosine ELISA was performed. Briefly, serum starved cells were pretreated with 50 μl/well DMEM containing 0.1% BSA and the indicated inhibitors for 30 minutes at 37° C. This was followed by stimulation of the cells with either 3 nM EGF or 1 nM NRG1β1 for 10 minutes at 37° C. Then the plates with cells were placed on ice, washed once with ice-chilled PBS, and lysed with a lysis buffer containing phosphatase inhibitor cocktails. Cell lysates were clarified by incubation with 20 μl of Protein-A-Sepharose bead slurry overnight at 4° C. on a plate shaker. The beads were then removed and the supernatant was used for phosphotyrosine ELISA. The HER1 and HER3 capture antibody plates for ELISA were prepared as follows: the 96-well assay plates were coated with 0.4 μg/mL anti-human EGFR antibody (#AF231) or with 4 μg/mL anti-human ErbB3 DuoSet IC (#DYC1769). Coated plates were blocked with 2% bovine serum albumin and 0.05% Tween-20 in PBS for 2 hours at RT. Cell lysate (75 μl) processed as above was transferred to each well of the coated plates, incubated overnight at 4° C. with mixing, and then washed 4 times with WB. Tyrosine phosphorylation on HER proteins was detected with 100 μl/well of an anti-phosphotyrosine-HRP conjugate (R&D Systems) diluted according to the manufacturer's instructions in PBS containing 2% BSA, and incubated for 2 hours at RT. The plates were washed 4 times with WB, and then developed with 100 μl/well TMB substrate followed by 100 μl/well stop reagent for TMB (both from Sigma-Aldrich). Color development time was varied so that the optical densities of the developed plates ranged from 0.5-1.0. The plates were read by a VERSAmax microplate reader (Molecular Devices, Sunnyvale, Calif.) at 650 nM.

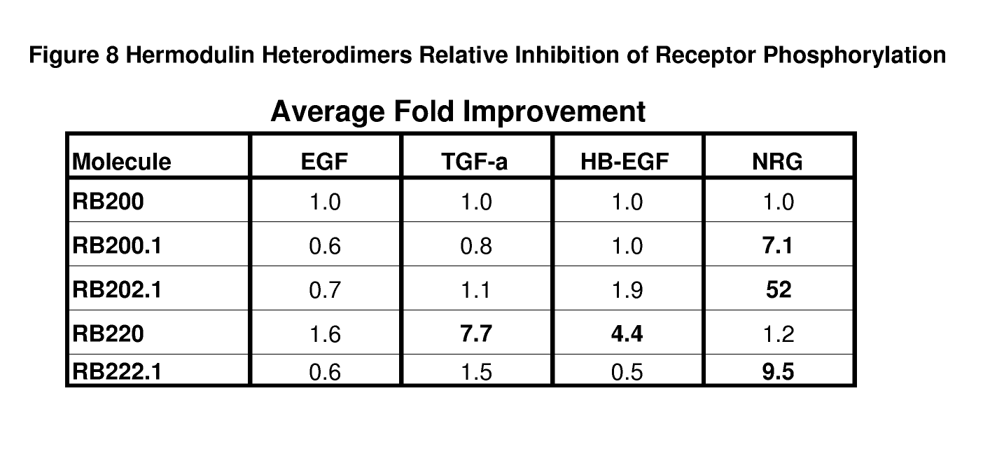

FIG. 8 shows the results of these experiments testing for relative inhibition of receptor phosphorylation by Hermodulin heterodimers. As shown in FIG. 8, there is no difference between the heterodimers for EGF. For TGF-a: RB220 is most effective while for HB-EGF, there is minimal difference between heterodimers. For NRG, RB202.1 is most potent, while RB200.1 and RB222.1 are more effective than RB200 or RB220.

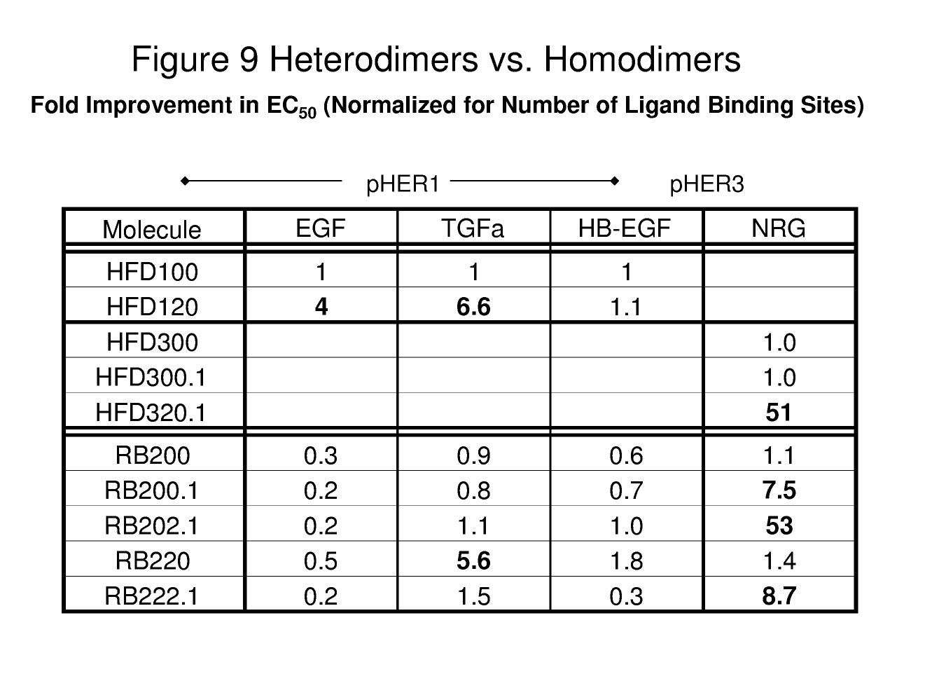

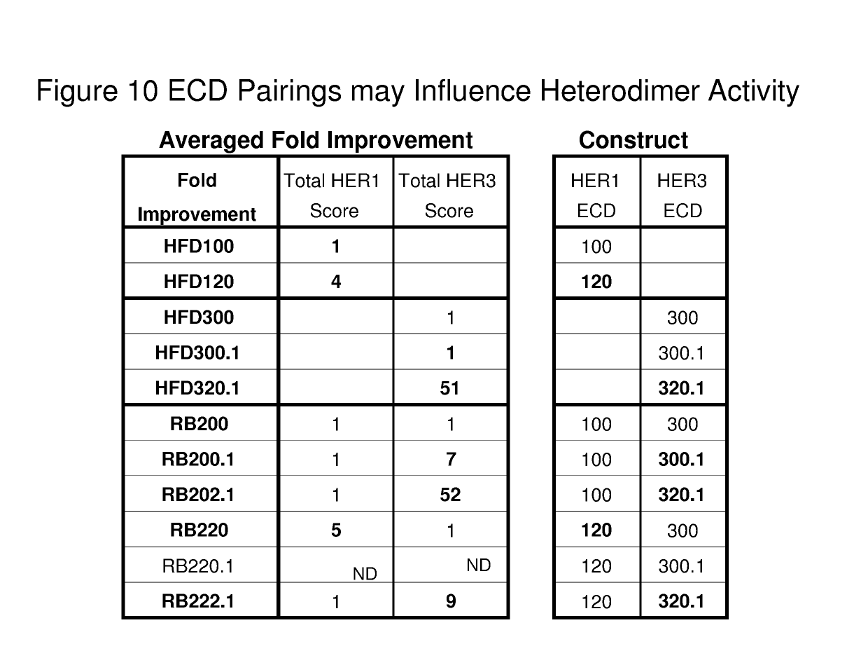

When normalized for the number of ligand-binding sites, then the results are shown in FIG. 9 where heterodimers are compared to homodimers. The table shows the fold improvement in EC₅₀ when the calculations are normalized for number of ligand binding sites. Unexpectedly, these results show that HFD320.1 sequence is fifty time more active than HF300 when paired with HFD100 and is similar to HF300.1 when paired with HF120. HFD 100 sequences not affected by the dimerization partners while HFD 120 sequence activity is attenuated when paired with HFD320.1 as compared to HFD300. HFD300.1 was not tested. As such, the results indicate that for various ECD pairings, the combination of the pairings may influence heterodimer activity. FIG. 10 show the results of average fold improvement for various ECD pairings that show that the pairings may influence heterodimer activity.

FIG. 17 (middle panel) shows that RB242 is more potent in inhibiting GF-dependent HER phosphorylation than RB 200.

### Example 8

### Hermodulin Inhibition of NRG-Induced Cell Proliferation

Different cell lines were used to test for Hermodulin's effect on ligand-induced proliferation. Cell proliferation studies were conducted in serum-free medium. Cells were plated in 96-well tissue culture plates (Falcon #35-3075, Becton Dickinson, NJ) at 2000 to 5000 cells per well in 100 μl culture medium, as appropriate for a cell line, and then grown overnight (15 to 18 hours). The cells were then serum-starved for 24 hours and were treated with 3 nM of EGF or NRG113 in the presence of increasing concentration of the indicated inhibitors for 3 days. Cell proliferation was quantified by the MTS assays. The plate was then read on a plate reader at 490 nm wavelength for absorbance, which was directly proportional to the amount of cells in the well.

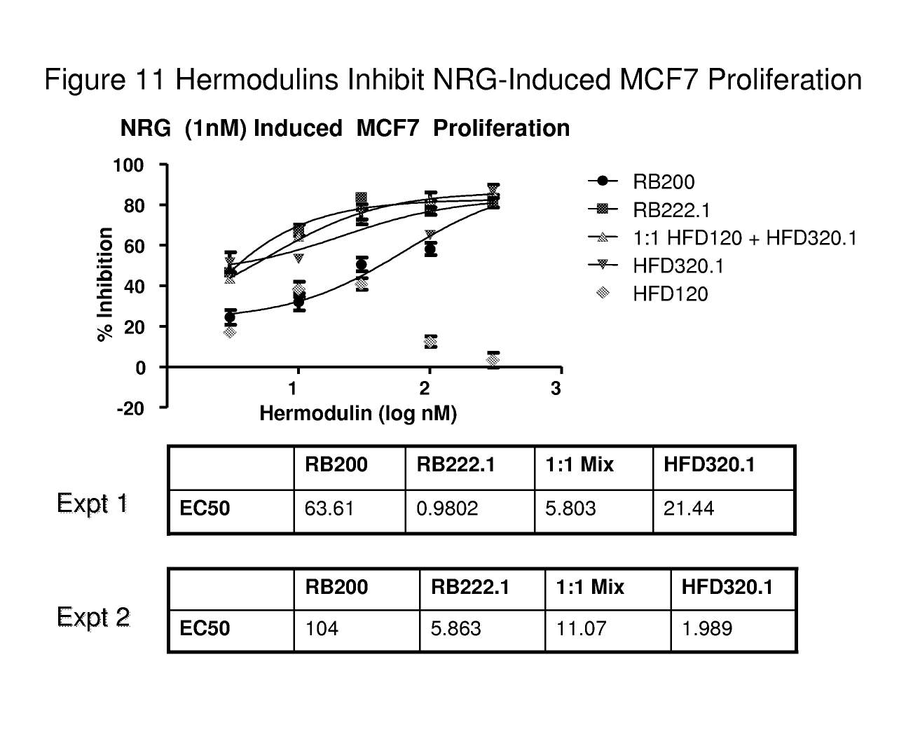

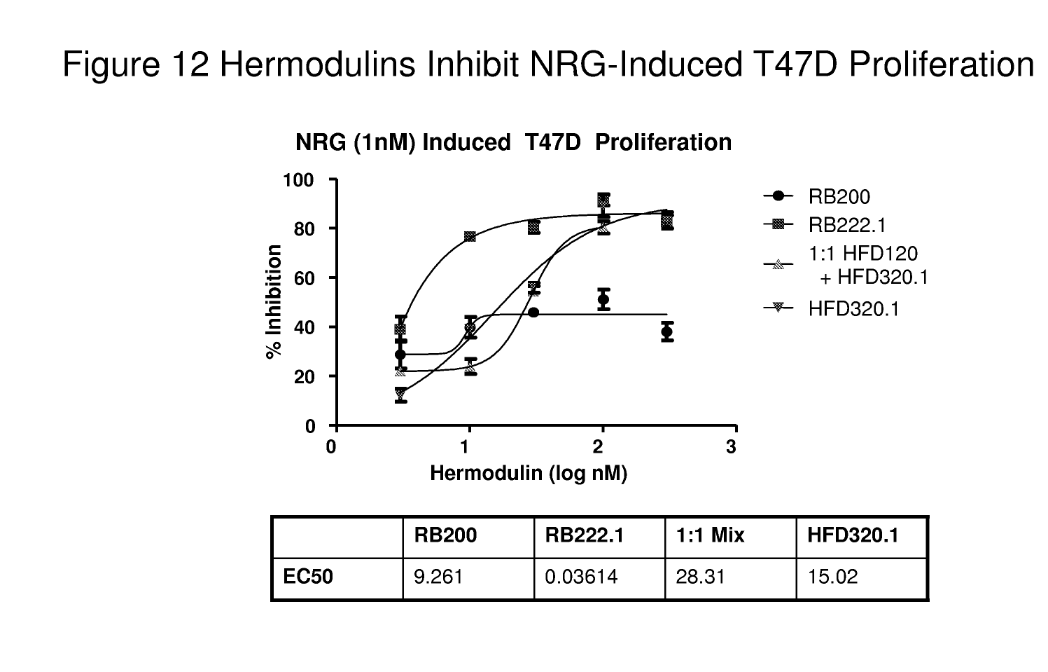

FIG. 11 shows the result of experiments testing for inhibition of NRG-induced MCF7 proliferation while FIG. 12 shows the result of experiments testing for inhibition of NRG-induced T47D proliferation. For NRG-induced proliferation, RB222.1 worked the best, followed by HFD320.1 and 1:1 Mix. RB200 was the least effective of the group for its effect on NRG-induced cell proliferation, although it does inhibit EGF-induced proliferation of MCF7 cells.

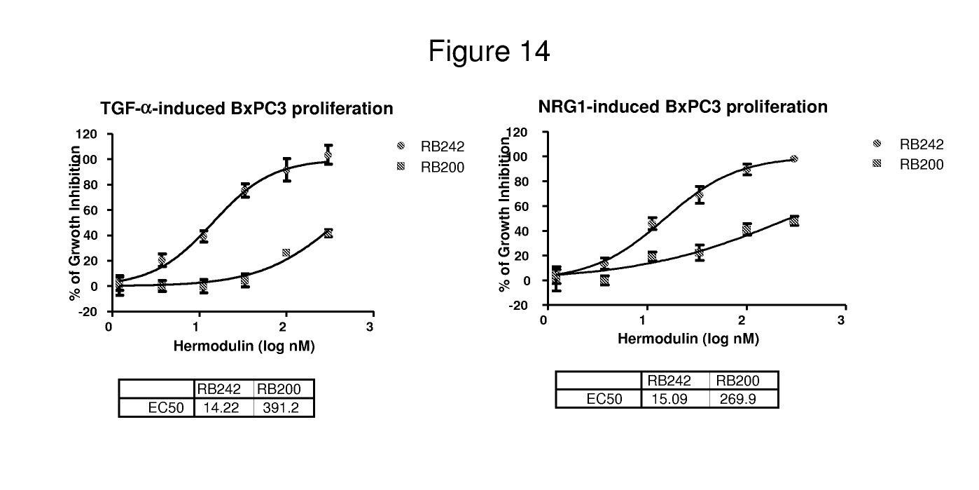

FIG. 14 shows that Hermodulin can inhibit ligand-induced cell proliferation. BxPC3 pancreatic cancer cells were treated with 3 nM of TGF-α (A) or 3 nM of NRG1-β1 (B) in the presence of increasing amounts of RB200 or RB242 for 3 days. Cell proliferation was quantified by MTS assay. The data are expressed as percent inhibition of cell growth as compared with the control cells stimulated with TGFα or NRG1-β1 alone. Data are mean±SEM of 8-replicate samples. FIG. 17 (bottom panel) also shows that RB242.1 is a potent inhibitor of GF-induced cell proliferation.

### Example 9

### Pharmacokinetic Analysis of Hermodulins

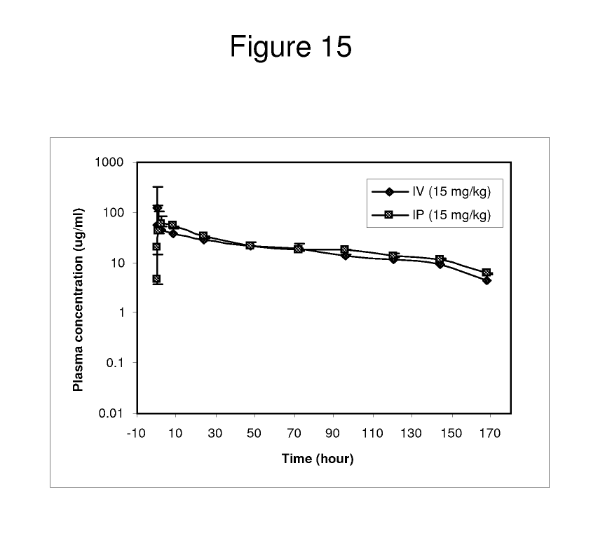

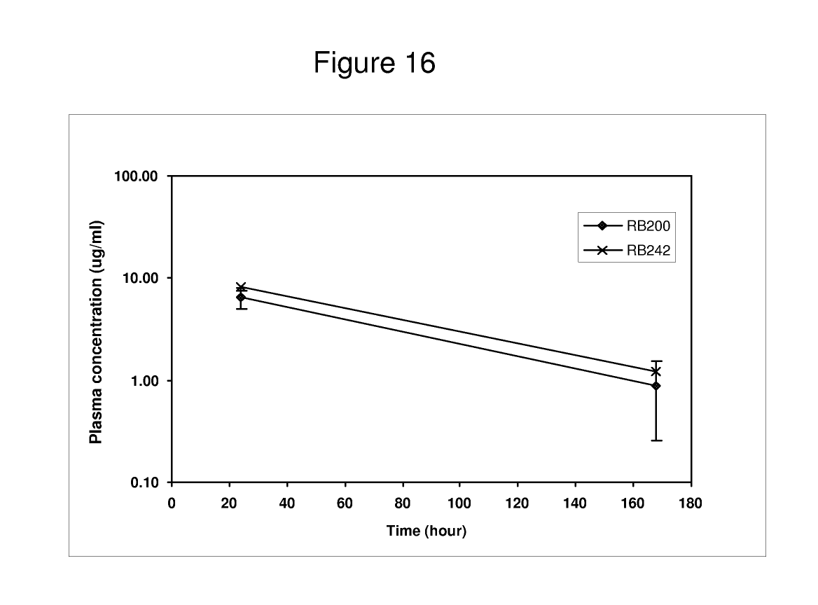

Plasma concentrations in rodent models of all Hermodulin constructs, including those with optimized Her3 were analyzed by a Hermodulin-specific ELISA, which use anti-HER1 (AF231, R&D System) and anti-HER3 (AF234, R&D Systems) antibodies as the capture, HRP conjugated anti-human Fc antibody (Bethyl Laboratories) as the reporter to show the extent of the administered dose that reaches the systemic circulation intact. Bioavailability, clearance rate and plasma half-life were then calculated. For RB200, the absolute bioavailability of RB200 measures the availability of RB200 in systemic circulation after IP administration of 15-30 mg/kg in mice by using the formula:

$$F = \frac{( AUC ) IP * doseIV}{( AUC ) IV * doseIP}$$

Where AUC=area under the curve. From this calculation, the estimated F_{RB200h} was determined to be >90%. Besides high bioavailability, RB200 also exhibited a low volume of distribution, and a prolonged terminal half-life consistent with expectations for Fc-fusion proteins and other therapeutic monoclonal antibodies. The calculations for other Hermodulins are done in the same manner to determine bioavailability and terminal half-life. FIGS. 15 and 16 show the plasma concentrations of various Hermodulins in rats and nude mice and the calculated pharmacokinetic parameters.

### Example 10

### Optimization of Her1 and Her3

The following tables illustrate some of the designed mutations that were tested for Her1 and binding activity to its cognate ligand.

| EGFR (Her1) | | Binding |
|---|---|---|
| Mutation | Sub-domain | Activity |
| Q8P | I | Decreased |
| S11L | I | Decreased |
| T15S | I | Increased |
| T15E | I | No binding |
| T15Y | I | No binding |
| T15Y, Q16E | I | No binding |
| T15K | I | No binding |
| Q16E | I | Decreased |
| Q16S | I | Not secreted |
| Q16K | I | Increased |
| Q16Y | I | No binding |
| Q16Y, G18D | I | Not secreted |
| L17V | I | Not secreted |
| L17I | I | Similar |
| G18N, D22E | I | No binding |
| G18N, T19G | I | Decreased |
| G18N, T19G, F20Y | I | No binding |
| G18N, T19N, F20Y | I | Decreased |
| T19D, F20A | I | No binding |
| T19D, F20A, D22N, | I | No binding |
| H23Q | | |
| T19D, F20A, D22N, | I | Not secreted |
| H23Q, F24Y | | |
| T19K | I | Increased |
| T19Q | I | Increased |
| T19D | I | Not secreted |
| T19Y | I | Decreased |
| T19G | I | Similar |
| T19I | I | Decreased |
| D22N | I | Increased |
| L25A | I | Similar |
| L25A, S26L | I | No binding |
| L25A, S26Q | I | Decreased |
| L25Y, S26A | I | Decreased |
| L25N, S26A | I | Not secreted |
| L25Q | I | Not secreted |
| S26L | I | Decreased |
| S26A | I | Decreased |
| S26T | I | Not secreted |
| M30L | I | Similar |
| N32E | I | Similar |
| T44V, Y45L | I | Decreased |
| T44V, Y45L, V46T, | I | Not secreted |
| Q47G | | |
| Y45W | I | Increased |
| L69V | I | Decreased |
| L69I | I | Similar |
| T71E, V72F, E73S, | I | Not secreted |
| R74T | | |
| V72F | I | Not secreted |
| Q81R | I | Similar |
| N86T, M87Q, Y88V | I | Similar |
| Y89H, E90D | I | Decreased |
| Y89W | I | Similar |
| L98M, S99L | I | Not secreted |
| L98M, S99F, D102N | I | Not secreted |
| S99A | I | Increased |
| S99T | I | Decreased |
| P112R, M113L, | I | Similar |
| R114T | | |
| F126I, S127E, N128K | I | Not secreted |
| F126I | I | Similar |
| F126I, N129K | I | Not secreted |
| P130D, A131K | I | Decreased |
| S145R, S146G | I | Not secreted |
| F148R, L149D, S150A, | I | Increased |
| N151E, M152I, | | |
| S153V | | |
| M154V, D155K, | I | Similar |
| F160G, Q161D | | |
| Y246A | II | No binding |
| Y246M | II | No binding |
| Y246V | II | No binding |
| V350L | III | Increased |
| F352H | III | No binding |
| G354A | III | Not secreted |
| A385D | III | Increased |
| W386E | III | Not secreted |
| H409V, G410R | III | Decreased |
| G410R | III | Not secreted |
| N420D | III | Similar |
| S440L | III | Increased |
| G441R | III | Decreased |
| K463E | III | Decreased |
| K463Q | III | Similar |
| I467Q, S468K | III | Decreased |
| I467K | III | Not secreted |
| D563L | IV | Similar |
| D563P | IV | Similar |
| G564D | IV | Increased |
| G564S | IV | Increased |
| H566G | IV | Increased |
| H566V | IV | Increased |
| N579R | IV | Similar |
| V583E | IV | Decreased |

For Her3, some of the mutations that were tested are as follows:

| HER3 | | Binding |
|---|---|---|
| Mutation | Sub-domain | Activity |
| A8S | I | Similar |
| L14E | I | Increased |
| G16D | I | Decreased |
| G16K | I | Similar |
| S18T | I | No Binding |
| V19Q | I | No Binding |
| D22T | I | Similar |
| A23F | I | Increased |
| A23L | I | Decreased |
| N25 | I | Decreased |
| R36N | I | Increased |
| V47T | I | Decreased |
| L48Y | I | No Binding |
| G50E | I | Decreased |
| A53Y | I | Increased |
| V70E | I | Decreased |
| M72L | I | Increased |
| V86I | I | Increased |
| D93E | I | Similar |
| F96L | I | No Binding |
| M101F | I | Decreased |
| L102S | I | No Binding |
| N103K | I | No Binding |
| N105R | I | Similar |
| T106K | I | Increased |
| T106Q | I | Similar |
| N107D | I | Decreased |
| S109N | I | Decreased |
| H110F | I | Decreased |
| R113Q-Q114E | I | Decreased |
| R116H | I | Decreased |
| T121I, | I | Decreased |
| P165L | I | Decreased |
| Y129R | I | Decreased |
| K132N | I | Increased |
| 132K | I | Decreased |
| G215D | II | Decreased |
| Y246A | II | Increased |
| Y246P | II | Increased |
| Y246V | II | Increased |
| K248E | II | Decreased |
| Q252D | II | Increased |
| Q252E | II | Increased |
| P309R | II | Similar |
| E313N | III | Decreased |
| 322DS | III | No Binding |
| D325N | III | No Binding |
| G331K | III | Similar |
| L339N | III | Similar |
| N341D | III | Decreased |
| D343F | III | No Binding |
| D343H | III | No Binding |
| D343I | III | No Binding |
| D343L | III | No Binding |
| D343S | III | Decreased |
| N350H | III | Similar |
| N350R | III | Decreased |
| P353S | III | Decreased |
| H355N | III | Decreased |
| K356A | III | Decreased |
| P358E | III | Similar |
| P362S | III | Similar |
| Y377F | III | Decreased |
| N379L | III | Decreased |
| H386N | III | Similar |
| H388T | III | Similar |
| N389D | III | Decreased |
| S403V | III | Similar |
| L404K | III | Decreased |
| Y405Q | III | Decreased |
| Y405T | III | Decreased |
| N406H | III | Decreased |
| R407G | III | Similar |
| R407Q | III | Decreased |
| R407Y | III | Increased |
| F409L, L411 | III | Decreased |
| L411, L417Q | III | Decreased |
| L412A | III | Increased |
| L412Y | III | Decreased |
| M414V | III | Similar |
| K415S | III | No Binding |
| R434N | III | Decreased |
| Y436G | III | Decreased |
| Y436L | III | No Binding |
| S438H | III | Similar |
| S438T | III | No Binding |
| S438V | III | Similar |
| A439D | III | No Binding |
| R441S | III | Increased |
| Q442N | III | Similar |
| E460K | III | Similar |
| E460N | III | Increased |
| E461G | III | Similar |
| E461Q | III | Increased |
| L463H | III | Decreased |
| L463S | III | Decreased |
| D464H | III | Increased |
| D464K | III | Decreased |
| D464Q | III | Increased |
| D464V | III | Decreased |
| K466I | III | Increased |
| K466P | III | Decreased |
| K466T | III | Increased |
| H467D | III | Increased |
| H467G | III | Increased |
| C481R | III | No Binding |
| S487F | III | Similar |
| D562-565deL | IV | No Binding |
| G563F | IV | Decreased |
| G563L | IV | Decreased |
| G563Q | IV | No Binding |
| G563R | IV | Decreased |
| H565E | IV | No Binding |
| H565F | IV | Decreased |
| H565I | IV | Increased |
| H565Q | IV | Increased |
| S569R | IV | Similar |
| I581D | IV | Similar |
| K583E | IV | Similar |
| I581V | IV | Decreased |

### Example 11

### Optimized HER3:Fc Suppresses Ligand Binding by Optimized EGFR

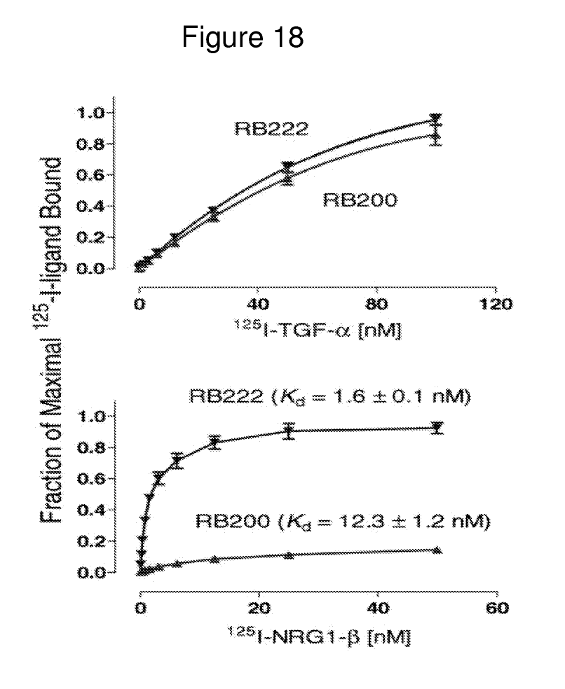

EGFR_{T15S}:Fc was co-expressed with HER3_{Y246A}: Fc in HEK293T cells, and the resulting heterodimer (RB222) was purified to ˜95% homogeneity. Ligand binding demonstrated that RB222 retained the improved affinity for ₁₂₅I-NRG1-β compared with the parent heterodimer RB200 (K_{d} of 1.6 nM versus 12.3 nM). However, RB222 no longer possessed the improved affinity for EGFR ligands. As shown in FIG. 18, heterodimers RB200 and RB222 each had an apparent K_{d}>30 nM for ₁₂₅I-TGF-a (binding was not saturated at 100 nM of ₁₂₅I-TGF-a), while the EGFR_{T15S}:Fc homodimer displayed a K_{d} of ˜1.0 nM for the same ligand. Thus, the HER3 ECD suppresses the high affinity binding of the EGFR ECD when they are locked in an Fc-mediated heterodimer.

### Example 12

### A G564S Mutation Restores the High-Affinity Binding of EGFR Ligand to RB222

To restore the high-affinity EGFR ligand binding to the heterodimer RB222, additional single mutations were introduced into the EGFR arm of RB222, focusing on its subdomain II/IV tether region. A method for efficient screening for EGFR ligand binding to the EGFR/HER3 heterodimer mutants in the conditioned media without prior purification was utilized. EGFR/HER3:Fc heterodimers as well as HER3:Fc homodimers in the conditioned media were immobilized on the surface of 96-well plates which were pre-coated with anti-human HER3 (ECD-specific) antibody. This was followed by binding of EGFR ligands to the immobilized EGFR/HER3:Fc heterodimers. An important advantage of this method is that conditioned medium containing a mixture of heterodimers and homodimers can be screened directly for the heterodimer-specific EGFR ligand binding without removal of the contaminating homodimers.

Ten heterodimer mutants were created and screened using this method. A mutant RB242 with a G564S mutation located in subdomain IV of the autoinhibitory tether was recovered which showed restored high-affinity EGFR ligand binding. RB242 was subsequently purified to ˜95% homogeneity and assayed for its ligand binding affinity.

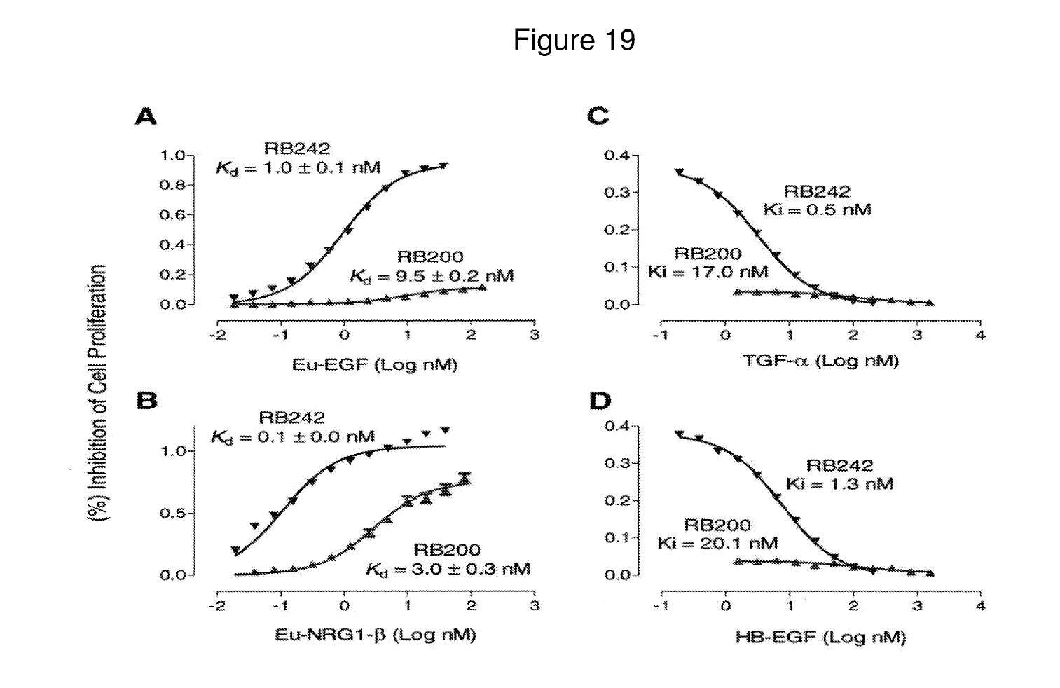

All initial ligand affinity screening performed above allowed for the procurement and comparison of the apparent K_{d} values. In order to determine the true K_{d}, the apparent K_{d} was used as a starting point to calibrate the saturation binding such that the concentration of an assayed receptor was at least 10-fold lower than the measured K_{d} for the assayed ligand. When binding assays were performed following this mathematic relationship, RB242 demonstrated a 10-fold improvement over RB200 in affinity for Eu-EGF (K_{d} of 1.0 nM versus 9.5 nM) and a 31-fold improvement in affinity for Eu-NRG1-β (K_{d} of 0.1 nM versus 3.1 nM, FIGS. 19A and B). Competitive ligand binding was performed to displace Eu-EGF binding by unlabeled TGF-a or HB-EGF. In these ligand displacement assays, RB242 demonstrated a 34-fold improvement over RB200 in affinity for TGF-a (Ki of 0.5 nM versus 17.0 nM), and a 16-fold improvement in affinity for HB-EGF (Ki of 1.3 nM versus 20.1 nM, FIGS. 19C and D).

Purified RB200 and RB242 were assayed for their ability to inhibit EGFR and HER3 phosphorylation. A dose-dependent inhibition of ligand-induced EGFR phosphorylation by RB200 or RB242 was demonstrated in N87 cells and MCF7 cells. As suggested by the increased ligand binding affinity, RB242 was 65-fold more potent than RB200 in inhibition of EGF-induced EGFR phosphorylation (EC₅₀ of 1.8 nM versus 117.3 nM) and 10-fold more potent in inhibition of TGF-a-induced EGFR phosphorylation (EC₅₀ of 19.4 nM versus 199.0 nM). Similarly, RB242 was 15-fold more potent than RB200 in inhibition of NRG1-β-induced HER3 phosphorylation in MCF7 cells (EC₅₀ of 1.7 nM versus 25.1 nM).

### Example 13

### RB242 is More Potent than RB200 in Inhibition of Proliferation of Cultured Tumor Cells

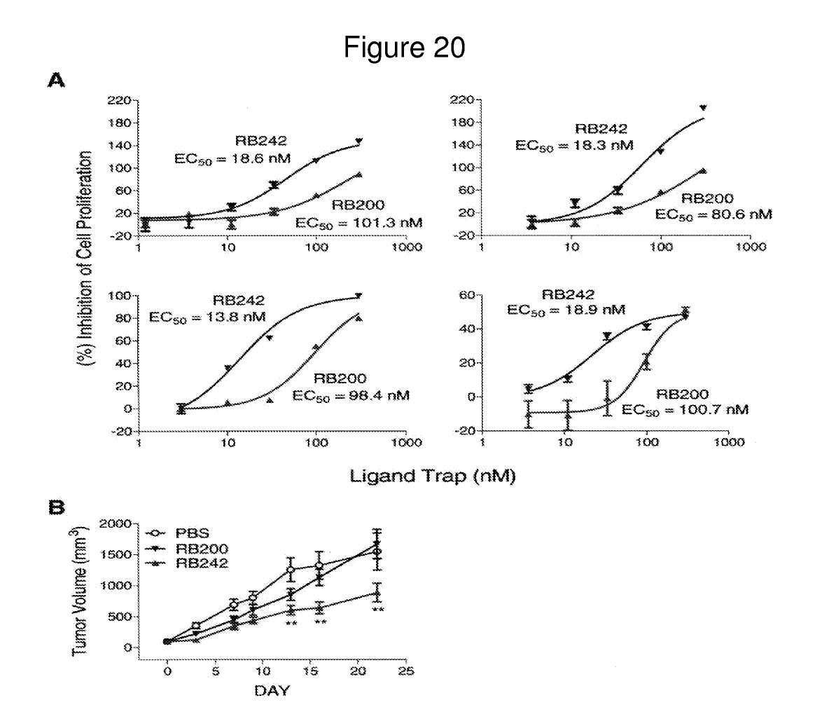

The effects of RB200 and RB242 on proliferation of cultured monolayer tumor cells were compared. Proliferation of BxPC3 pancreatic cancer cells was induced by TGF-a or NRG1-β in serum-free medium. Growth factor-induced BxPC3 proliferation was inhibited by RB200 or RB242 in a dose-dependent manner (FIG. 20A, top panels). The estimated EC₅₀ indicated that RB242 was ˜5-fold more potent than RB200 in inhibition of TGF-a- or NRG1-β-induced proliferation in a 3-day proliferation assay. As much as 200% inhibition was seen in RB242-treated BxPC3 cells. This presumably resulted from proliferation of BxPC3 cells in serum-free condition which was inhibited by RB242. Similarly, serum-starved MCF7 breast cancer cells were induced to proliferate by NRG1-β; this proliferation was inhibited by RB200 or RB242 (FIG. 20A, bottom left panel). The estimated EC₅₀ indicated that RB242 was 7-fold more potent than RB200 in a 5-day proliferation assay. Proliferation of human H1437 NSCLC cells was analyzed in growth medium (RPMI1640/10% FBS) with increasing concentrations of RB200 or RB242. As shown in FIG. 20A (bottom right panel), RB242 was about 5-fold more potent than RB200 in a 5-day proliferation assay (EC₅₀ of 18.9 nM versus 100.7 nM).

### Example 14

### RB242 Demonstrates Improved Anti-Tumor Activity in a Mouse Model of Human Non-Small Cell Lung Cancer

In vivo efficacy of RB200 and RB242 was compared in nude mice bearing tumors derived from human H1437 NSCLC cells. Mouse tumor xenograft model used: the H1437 non-small cell lung cancer (NSCLC) tumor xenograft study was performed in female CD-1 nu/nu nude mice. Efficacy studies were done in groups of 9 mice. H1437 cells were maintained in RPMI 1640/10% FBS. Cells were harvested with 0.025% EDTA, washed twice with culture medium, resuspended in sterile PBS, and then injected subcutaneously into mice at 6×10₆ cells in 100 μl volume. Tumor measurements were done using a caliper, and tumor volume was calculated from length, width, and cross sectional area. Treatment began when the mean tumor volume reached approximately 100 mm₃. Mice were dosed with RB200 or RB242 at 12 mg/kg i.p. in 150 μl volume, 3 times weekly for three weeks. Experiment was carried out under the regulatory guidelines of OLAW Public Health Service Policy on Humane Care and use of Laboratory Animals (1996), the policies set forth in the Guide for the Care and Use of Laboratory Animals, and under the IACUC of the Palo Alto Medical Foundation. The results from mouse tumor xenograft experiment were analyzed using 2-way ANOVA with Bonferroni's post-test.

This mouse tumor model was chosen in part because RB200 and RB242 showed direct antiproliferative activity in vitro (FIG. 20A bottom right). H1437 cells were injected subcutaneously and allowed to grow to ˜100 mm³ before treatment started. In this model, RB200 dosed at 12 mg/kg showed a trend in growth inhibition of the established tumors (FIG. 20B; P>0.05). Administered at the same dose, RB242 demonstrated improved anti-tumor activity with ˜50% inhibition of tumor growth after two weeks of treatment (P<0.01), consistent with its enhanced inhibitory activity in cultured tumor cells (FIG. 20A).

1. A multimer comprising an extracellular domain (ECD) from Her3 which has been optimized to improve binding to its cognate ligand.

2. The multimer of claim 1 wherein the Her3 ECD comprises a Y246A mutation.

3. The multimer of claim 2 wherein the Her3 ECD comprises a lysine at position 132.

4. The multimer of claim 1 wherein the Her3 ECD comprises a K132E mutation.

5. The multimer of claim 1 wherein the Her3 ECD is truncated.

6. The multimer of claim 1 further comprising an ECD from Her1.

7. A multimer comprising an extracellular domain (ECD) from Her1 that has been optimized to improve binding to its cognate ligand.

8. The multimer of claim 7 wherein the Her1 ECD comprises a T15S mutation.

9. The multimer of claim 8 further comprising a G564S mutation.

10. The multimer of claim 9 further comprising a Her3 ECD comprising a Y246A mutation.

11. The multimer of claim 7 wherein the Her1 ECD comprises a domain 4 deletion.

12. The multimer of claim 7 wherein the Her1 ECD comprises one or mutations selected from the group consisting of S193N, E330D, and G588S.

13. A composition comprising a Her1 homodimer wherein the Her1 has been optimized to improve binding to its cognate ligand.

14. The composition of claim 13 wherein the Her1 comprises one or more mutations selected from the group consisting of: T15S, G564S, domain 4 deletion, S193N, E330D, and G588S.

15. The composition of claim 13 wherein the Her1 comprises T15S and G564S mutations.

16. A composition comprising a Her3 homodimer wherein the Her3 has been optimized to improve binding to its cognate ligand.

17. The composition of claim 16 wherein the Her3 comprises a Y246A mutation.

18. A composition comprising a heterodimer of a Her3 variant and a Her1 variant wherein each variant has been optimized to improve binding to its cognate ligand.

19. The composition of claim 18 wherein the Her1 variant comprises one or more mutations selected from the group consisting of: T15S, G564S, domain 4 deletion, S193N, E330D, and G588S.

20. The composition of claim 18 wherein the Her1 variant comprises T15S and G564S mutations.

21. The composition of claim 18 wherein the Her3 variant comprises one or both mutations selected from the group consisting of: Y246A and K132E.

22. The composition of claim 20 wherein the Her3 variant comprises a Y246A mutation.

23. The composition of claim 13, which additionally comprises Fc receptor linked to Her1.

24. A composition comprising a mixture of Her1/Her1 homodimers, Her1/Her3 heterodimers and Her3/Her3 homodimers wherein the Her1 and/or the Her3 components have been optimized to improve ligand binding.

25. The composition of claim 24 wherein the Her1 variant comprises one or more mutations selected from the group consisting of: T15S, G564S, domain 4 deletion, S193N, E330D, and G588S and wherein the Her3 variant comprises one or both mutations selected from the group consisting of: Y246A and K132E.

26. The composition of claim 24 wherein the Her1 variant comprises T15S and G564S mutations and wherein the Her3 variant comprises a Y246A mutation.

27. (canceled)

28. A method of inhibiting the growth of a cancer cell comprising contacting the cell with a composition comprising a Her1 variant and a Her3 variant wherein the Her 1 and/or the Her3 components have been optimized to improve ligand binding.

29. The method of claim 28 wherein the Her1 variant comprises T15S and G564S mutations and wherein the Her3 variant comprises a Y246A mutation.

30. A method of reducing the volume of a tumor comprising contacting the cell tumor with a composition comprising a Her1 variant and a Her3 variant wherein the Her 1 and/or the Her3 components have been optimized to improve ligand binding.

31. The method of claim 30 wherein the Her1 variant comprises T15S and G564S mutations and wherein the Her3 variant comprises a Y246A mutation.
